(19) United States
(12) Patent Application Publication
(10) Pub. No.: US 2006/0035981 A1
(43) Pub. Date: Feb. 16, 2006

(54) Inhibition of anaerobic glucose metabolism and corresponding composition as a natural non-toxic approach to cancer treatment

(76) Inventors: Elizabeth Anne Mazzio, Tallahassee, FL (US); Karam F. Soliman, Tallahassee, FL (US)

Correspondence Address: Karam Soliman;Florida A & M University, College of Pharmacy and Pharmaceutical Sciences, 104 Dyson Building, Tallahassee, FL, 32307, US

(21) Appl. No.: 11/233,279

(22) Filed: Sep. 20, 2005

Related U.S. Application Data
(63) Continuation-in-part of application No. 10/909,590, filed on Aug. 2, 2004, now abandoned.
(60) Provisional application No. 60/491,841, filed on Aug. 2, 2003.
(60) Provisional application No. 60/540,525, filed on Jan. 29, 2004.

(51) Int. Cl. A61K 31/12; A61K 31/7072; A61K 31/7076; A61K 31/525; A61K 36/328; A61K 36/23; A61K 36/906

(52) U.S. Cl. 514/690; 514/45; 514/51; 514/27; 514/251; 424/725; 424/748; 424/756; 424/745; 424/746; 424/729

(57) ABSTRACT

This invention discloses a method and formulation for treatment/prevention of human and animal cancers. The invention is designed to exploit the vulnerability of cancer with regards to its anaerobic requirement for non-oxidative phosphorylation of glucose to derive energy, which is opposite to the host. The composition is comprised of a combination of one or more of (A) 2,3-dimethoxy-5-methyl-1,4-benzoquinone, ubiquinones (5-45) (B) compound(s) capable of augmenting oxidative phosphorylation such as a riboflavin containing compound and/or ubiquinone (50) (C) 2′,3,4′5,7-pentahydroxyflavone or a lactic acid dehydrogenase inhibitor and (D) compounds (s) that antagonize gluconeogenesis from non-glucose carbon based substrates. The combination of these substances should favor oxidative loss of carbon through decarboxylation reactions, suppress gluconeogenesis and initiate collapse of glycolysis in tumor tissue, a chemical manipulation that should be non-toxic or perhaps even beneficial to normal respiring host tissue. Pilot studies indicate the treatment to be effective without side effects.

### CROSS-REFERENCE TO RELATED APPLICATIONS

This application is a continuation in part of application No. U.S. Pat. No. 0,527,403 filed on Aug. 5, 2005 and application Ser. No. 10/909,590 filed on Aug. 2, 2004, which claims the benefit under 35 USC 119(e), of previous application(s) No. 60/491,841 filed on Aug. 2, 2003 and No. 60/540,525 filed on Jan. 29, 2004, all of which are herein incorporated by reference.

### STATEMENT REGARDING FEDERALLY SPONSORED RESEARCH AND DEVELOPMENT

The U.S. government has certain rights to this invention as federal support was provided for by NIH Grant NCRR 03020.

### FIELD OF THE INVENTION

The present invention describes a composition and method for treatment and/or prevention of human and animal cancers. The invention describes the use of compounds that increase oxygen utilization and impair anaerobic metabolism of cancer cells, an event that corresponds to selective cancer cell death. The invention therefore, relates to the fields of pharmacology, oncology, medicine, medicinal chemistry and biochemistry.

### DESCRIPTION OF THE RELATED ART

Current chemotherapy for cancer often employs the use of drugs that are generally toxic to the body as well as the tumor. The efficacy of drugs are often in close proximity to systemic toxic effects, rendering a narrow therapeutic index. It is the objective of this invention to widen that gap by recognizing and exploiting a major difference between the host and the tumor with regards to how each derives energy (adenosine-5′-triphosphate (ATP)) from glucose. This invention delineates a natural formulation that should strategically starve the tumor by blocking anaerobic utility of glucose, while specifically augmenting aerobic energy metabolism of the host. The combined events should render non-toxic effects on the host and a detrimental adverse effect specific to the tumor. We initiated a diverse range of studies in order to elucidate the aberrant nature of glucose metabolism within malignant blastoma in vitro, and subsequently formulated a test composition which was found effective in arresting MD-MB-231 human mammary carcinoma in a xenograft model using Nu/Nu nude mice, comparable to paclitaxel (taxol®) in a pilot study. The pilot formulation did not appear to have any adverse effect on the animal's health as evident by absence of change in behavior, appetite, weight loss, food intake or excretory function. Moreover, the treatment was also effective in its water-soluble form and may be altered to suit a range of solubilities, thereby eliminating requirements for emulsifying agents or solvent vehicles that can at times lead to complication associated with chemotherapy such as hypersensitivity reactions.

For decades, it has been widely known that the etiological pathogenesis of cancer involves an inherent but unclear abnormality of glucose metabolism, one that is divergent from typical oxidative metabolic processes of eukaryotic cells. The “Warburg effect” is a common terminology used to describe the observed high glycolytic activity that occurs in cancer cells, even in the presence of low oxygen (“O₂”) concentration. Otto Warburg was a pioneer in demonstrating that cancer cells exhibit merged basal features having high capacity anaerobic and aerobic metabolic pathways. Since then, subsequent studies have consistently corroborated the inherent nature of cancer to involve a) rapid consumption of glucose b) robust glycolytic activity (Maublant et al., Bull Cancer, 85:935-50, 1998), c) rapid cell proliferation (Chesney et. al, Proc Natl Acad Sci USA, 96:3047-52, 1999), d) production and accumulation of lactic acid (Baggetto, Biochimie, 74:959-74, 1992) and e) a low extracellular pH with depleted glucose levels circumscribing the perimeter of the tumor. Our baseline findings are consistent with these experimental observations (Mazzio et al., Brain Res. 2004 Apr. 9; 1004(1-2):29-44) indicating that the predominant source of energy in the form of ATP is being produced primarily through anaerobic substrate level phosphorylation in the cytoplasm, even in the presence of what we found to be functional mitochondria. Further, evidence that suggests carbon dioxide (“CO₂”) can contribute to the acidity of a tumor (Helmlinger et al., Clin Cancer Res., 8:1284-91, 2002; Griffiths et al., Novartis Found. Symp., 240:46-62; 62-7,152-3, 2001), and O₂ deprivation/tissue hypoxia exacerbates the growth of cancer and resistance to chemotherapy (Brizel et al., Int. J. Radiat. Oncol. Biol. Phys., 51:349-53, 2001; Brizel et al, Int. J. Radiat. Oncol. Biol. Phys., 38:285-290, 1997; Alagoz et al., Cancer 75:2313-22, 1995), both clearly indicate that cancer has a preference for CO₂ and a low requirement/preference for lack of O₂. We conjecture that if the low requirement for O₂ involves a non-requirement for mitochondrial oxidative phosphorylation (OXPHOS) to derive energy from glucose, this vulnerability could possibly be exploited in order to kill the cancer without harm to the body.

Basically, our initial studies in vitro, demonstrate that cancer cells a) readily survive without O₂ b) prefer CO₂ and c) that changes in O₂/CO₂ are intricately involved with the way in which cancer cells metabolize glucose which subsequently control either cell death or cell viability/proliferation. Moreover, experimental block of mitochondrial respiratory function (e.g. with use of mitochondrial monocarboxylic pyruvate transport blocker, toxins such as 1-methyl-4-phenylpyridinium (MPP+), rotenone), the absence of O₂ or a high concentration of CO₂, are all conditions that can prompt a robust potentiation of glucose metabolism through glycolysis, while having no toxic effects other than depletion of glucose supply in a glucose-limited environment (Mazzio and Soliman, Biochem Pharmacol. 67:1167-84, 2004; Brizel et al., Int. J. Radiat. Oncol. Biol. Phys., 51:349-53, 2001; Brizel et al, Int. J. Radiat. Oncol. Biol. Phys., 38:285-290, 1997). These findings suggest cancer cells are completely anaerobic in nature, while having expendable oxidative apparatus. In contrast, we found that an increase in the concentration of O₂, a drop in the CO₂/O₂ ratio, or potentiating the function of the mitochondria all render collapse of substrate level phosphorylation (anaerobic ATP production from glucose) in cancer cells, an event that corresponds to cell death. These results indicate that tumor cells are not only anaerobic in nature, but react adversely to substrates or substances that augment aerobic mitochondrial function. And, there is an inverse opposing force between anaerobic and aerobic systems within the same cell. Further, these findings suggest that glucose metabolism in cancer is in direct opposition to the host, which favors aerobic conditions, where enhanced mitochondrial function is beneficial, mitochondrial toxins are poisonous and a high concentration of CO₂ can lead to suffocation through the halt of mitochondrial energy production. This metabolic anomaly we found to be consistent amongst cancer cells of various origin such as human, rat and mouse.

From this, we conjectured that if O₂ (a substrate for complex IV of the electron transport chain) is toxic to cancer cells, where the loss of complex IV function (via MPP+) is beneficial to cancer cells in terms of metabolic activation and glucose metabolism, then would it suffice to say that other mitochondrial substrates that serve to augment respiratory function be detrimental to glucose metabolism within cancer cells? And, if this event is further combined with direct selective inhibition of lactic acid dehydrogenase enzyme activity (LDH), could this block glucose utilization specifically in cancer cells? The combined manipulation should, in theory, yield greater concentration of pyruvate to fuel aerobic oxidative function by blocking its ability to drive substrate level phosphorylation through LDH. In brief summary, our findings demonstrate that a kinetic potentiation of the Vₘₐₓ and reduction of Kₘ of mitochondrial complex I can yield a robust enhancement of O₂ utilization through cytochrome oxidase (complex IV) in cancer cells. This event alone, is analogous to high O₂ concentration, and creates a significant vulnerability by impairing the use of glucose to produce ATP through substrate level phosphorylation (Mazzio and Soliman, Biochem Pharmacol. 67:1167-84, 2004). If anaerobic metabolism is further blunted by downregulation/inhibition of cytosolic LDH, a block of anaerobic glucose utilization occurs, corresponding to selective tumor cell death through loss of energy production (not yet published). Lastly, our research also suggests a component of anaerobic glucose metabolism in cancer cells—that includes a robust capacity for gluconeogenesis from non-glucose carbon based substrates and variants of carboxylation reactions, with potential roles for the following enzymes: acetate-coA ligase, malate synthase, isocitrate lyase, aconitase, phosphoenolpyruvate carboxylase/carboxykinase, glycolate oxidase, phosphoglycolate phosphatase, glycolaldehyde dehydrogenase, pyruvate carboxylase, citrate lyase, ferridoxin oxidoreductase, fructose 1,6-bisphosphatase, 2,3-diphosphoglycerate mutase, propionyl CoA carboxylase, malic enzyme, acetyl CoA carboxylase and ribulose-1,5-bisphosphate carboxylase, even though some of these are not known to be inherent to tumor tissue. The invention is a composition and means to specifically block glucose metabolism in cancer cells by combining agents that 1) inhibit LDH (without affecting glycolysis) 2) inhibit anapleurotic gluconeogenic pathways and 3) optimize aerobic metabolism by potentiating mitochondrial function.

While there has been relatively little to no research investigating the optimization of mitochondrial function or inhibition of anapleurotic gluconeogenic pathways to downregulate the growth of cancer, the block of ATP production through substrate level phosphorylation by inhibition of LDH in hindsite appears to be an obvious target. LDH plays a critical role in directing aggressive malignancies (Walenta and Mueller-Klieser. Semin Radiat Oncol 2004; 14:267-74), and its enzyme function is required to generate NAD+ as an enzymatic product and cofactor for glyceraldehyde 3-phosphate dehydrogenase which propels ATP production through phosphoglycerate/pyruvate kinase. Interestingly, while it may seem implausible that direct LDH inhibition would not render the body harm, we have screened a number of compounds and our research suggests that the most powerful anti-cancer flavonoids commonly consumed and sold over the counter, also inhibit the activity of LDH (LDH-5 (M₄)) (not yet published), an enzyme most resembling that inherent to human cancer (Koukourakis et al., Br J Cancer. 2003; 89:877-85; Augoff and Grabowski. Pol Merkuriusz Lek 2004; 17:644-7; Nagai et al., Int J Cancer. 198815;:10-6; Evans et al., Biol. Chem. 1985; 260:306-14). Yet, this has not yet been investigated as a likely avenue by which plant derived compounds exert well known anti-cancer effects (Rosenberg et al., J Chromatogr B Analyt Technol Biomed Life Sci. 777: 219-32, 2002; Stoner and Mukhtar, J Cell Biochem Suppl. 22:169-80, 1995). Prior research defining tumoricidal effects of flavonoids have focused on cell signaling, inhibition of protein kinase, tyrosine kinase, cyclin-dependent kinases, cell cycle phases G0/G1 or G2/M, proliferation or induction of apoptosis (Faderl and Estrov, Leuk Res. 27, 471-3, 2003;. Agullo et al., Biochem Pharmacol. 53:1649-57, 1997). Our results indicate that specific flavonoids can directly inhibit LDH possibly by oxidation of thiol group function, thereby impairing the catalytic region of the enzyme. And, if this is so, the loss of ATP through substrate level phosphorylation as a result of LDH inhibition would render a cavalcade of the observed down stream apoptotic events. The importance of LDH itself, in the progression of cancer has been substantiated, albeit relatively sparsely considering the magnitude of its role, in the literature (Shim et al., Proc Natl Acad Sci USA. 94:6658-63. 1997; Sun et al., Zhonghua Zhong Liu Za Zhi 13:433-5, 1992). The downregulation of LDH in BGC-823 gastric carcinoma cells can induce tumoricidal effects (Yang et. al, Zhonghua Zhong Liu Za Zhi 18:10-2, 1996) and remission of cancer and survival rates in humans undergoing chemotherapy to platinum drugs corresponds to a diagnostic reduction in serum LDH (Velasquez et al., Blood 71:117-22, 1998). Interestingly, while studies demonstrate heightened LDH concentrations to be associated with aggressive malignancies, the use, synthesis or evaluation of LDH inhibitors to actually treat cancer have not yet been described in the research literature. Further, our findings suggest that LDH inhibition could be lethal to cancer, but not as detrimental to the host as one would be inclined to think. In this invention, LDH inhibition is a part of the optimal synergistic combination of chemicals that should signal collapse of anaerobic glycolysis in cancer cells while having potentially beneficial effects on aerobic metabolic activity of the host.

While the descriptive embodiment defines a new slant on the approach to treatment, with regards to the use of specific compounds to alter oxidative glucose metabolism through enzymatic modulation, the concept is in alignment with existing scientific findings. While the mechanism is not known as to why O₂ is toxic to cancer cells, studies are generally consistent in reporting a deprivation of O₂ (ie hypoxia) to be associated with potentiation of glycolysis (Nielsen et al., Cancer Res. 61:5318-25, 2001) and enhanced tumor proliferation (Brizel et al., Int. J. Radiat. Oncol. Biol. Phys., 38:285 290, 1997). Similarly, improved therapeutic efficacy has been observed using carbogen (95% O₂/5% CO₂) to augment radiotherapeutic response to transplanted rat GH3 prolactinomas (Robinson et al., Br J. Cancer, 82: 2007-14, 2000), and hyperbaric O₂ arrests the growth of tumors resistant to chemotherapy and potentiates the effects of cisplatin (Alagoz et al., Cancer 75:2313-22, 1995). While the previous research suggests a physical approach to capitalize on the difference between the host and cancer regarding concentration and supply of O₂, the embodied invention is by nature chemical, and achieves the same means by increasing the aerobic (O₂ requiring)/anaerobic glucose metabolic ratio. A pilot test composition was formulated in order to contain agents that could optimize aerobic mitochondrial function and promote greater yield of pyruvate toward the mitochondria, while suppressing anaerobic glucose utility and the ability of pyruvate to sustain substrate level phosphorylation through LDH (without affecting the remainder of the glycolytic pathway), an otherwise critical requirement for tumor cells to utilize glucose to produce ATP. The treatment was found to be effective in arresting MD-MB-231 human mammary carcinoma in a xenograft model using Nu/Nu nude mice comparable to taxol®, while having no adverse effects on the animals with regards to health, behavior, appetite or weight loss. Although preliminary, this suggests that the embodiments of the present invention may yield significant tumor suppression without toxicity to the host. Similarly, studies employing the use of individual chemicals that comprise this invention in non-analogous studies, do not report toxicity, death or adverse effects on animal models when administered up to 300 mg/kg (Knudsen et al., Free Radic Biol Med 1996:20(2):165-73; Chen et al., Free Radic Biol Med 1995:20(5):949-953). The chemicals and substances that mediate effects through the described mechanism and that comprise the formulation, can be purchased from chemical distributors and/or can be synthesized or extracted from natural substances, as described below.

Briefly, the formulation contains one or more compounds that synergistically promote oxidative metabolism and/or impede lactic acid dehydrogenase or anaerobic glucose metabolism. The formulation can contain 2,3-dimethoxy-5-methyl-1,4-benzoquinone (herein also termed “DMBQ”) (quinoid base), and options for the entire ubiquinone series including corresponding hydroquinones, ubichromenols, ubichromanols or synthesized/natural derivatives and analogues. Ubiquinone structures are designated by the number of isoprene units attached to the 2,3-dimethoxy-5-methyl-1,4-benzoquinone base, which designates the term “coenzyme Qn”. Ubiquinones are also defined as the number of carbon atoms comprising the side chain and termed “ubiquinone (x)” where x is (0-50+ carbon atoms) and each isoprenene unit constitutes a 5 carbon atom extension.

The embodiment of the invention establishes the short chain ubiquinones (CoQ<3) as anti-cancer agents. More specifically, 2,3-dimethoxy-5-methyl-1,4-benzoquinone (DMBQ) is in excess of 1000× more potent than CoQ10 as an anti-cancer agent, causing collapse of anaerobic glucose metabolism through a mechanism we are continuing to explore, possibly involving enzymes integral for gluconeogensis in converting non-glucose carbon based substrates into glucose. While there is a wealth of information describing the use of CoQ10, there is little known about the potential use for short chain ubiquinones. Of the few publications noted, CoQ0 has been described in an oral hygiene formulation owned by SmithKline (WO03037284, May 08, 2003, Hynes) and the use of coenzyme Q2, Q4, Q6 in a method for treating or preventing mitochondrial dysfunction associated with Friedreich Ataxia, hypertrophic cardiomyopathy, Hallervorden-Spatz disease and sideroblastic anemia (U.S. Pat. No. 6,133,322, Oct. 17, 2000, Rustin and Roetig). Coenzyme Q2 has been used as a component in a formulated treatment for dementia (JP4112823, Apr. 14, 1992, Imagawa) and Q9 has been described in combination with CoQ10 for poultry feed formulations (EP0913095, May 6, 1999, Aoyama and Sugimoto). There is also meager technical research investigating efficacy or use for DMBQ, other than a few studies that define it as protective effects against lipid peroxidation in kidney, liver, heart, lung and spleen tissue in animal models of oxidative injury (Knudsen et al., Free Radic Biol Med. 1996; 20(2):165-73; Chen and Tappel, Free Radic Biol Med. 1995 May; 18(5):949-53). On the other hand, structurally related derivatives of CoQ (e.g chloroquinones and alkylmercapto-1,4-benzoquinones) (Porter et al., Bioorganic Chemistry 1978: 7:333-350; Folkers et al., Res Comm Chem Path Pharm 1978: 19(3) 485-490; Wikholm et al., Journal of Med Chem 1974:17:893-896) and a range of structurally similar compounds (e.g. 2,5-diaziridinyl-3,6-bis (carboethoxyamino)-1,4 benzoquionone (U.S. Pat. No. 4,233,215, Nov. 11, 1990, Driscoll et al.,) and 6-methoxy-10-cis-heptadecene-1,4-benzoquinone (CN 1362061, Aug. 7, 2002, Dehua et al.,) have been described as anti-tumor agents.

On the other hand, there is abundant literature on CoQ1 which is widely known for its role as a cofactor in mitochondrial enzymes that carry out oxidation-reduction reactions involved with aerobic ATP production. Our studies suggest that CoQ10 can increase the Vₘₐₓ of mitochondrial complex II activity in cancer cells (Mazzio and Soliman, Biochem Pharmacol. 67:1167-84, 2004) however, this did not control the rate of mitochondrial respiration or O₂ utilization through complex IV. And, we did not find CoQ10 to be as lethal as expected. Likewise, previous studies that have employed CoQ10 against cancer have been somewhat contradictory. For example, several reports show a positive inverse correlation where low physiological Q10 concentrations are associated with greater risk for cancer (Palan P R et al., Eur J Cancer Prev. 2003 August; 12(4):321-6; Portakal et al., Clin Biochem. 2000 June; 33(4):279-84; Jolliet P et al., Int J Clin Pharmacol Ther. 1998 September; 36(9):506-9) and its administration induces tumoricial effects (Gorelick C et al., Am J Obstet Gynecol. 2004 May; 190(5):1432-4), blocks the growth of cancer (Lockwood K et al., Biochem Biophys Res Commun. 1995 Jul. 6; 212(1):172-7; Lockwood et al., Biochem Biophys Res Commun; 1994 Mar. 30; 199(3)1504-1508; Folkers et al., Biochem Biophys Res Commun 1993 Apr. 15; 192(1) 241-245) and reduces side effects of chemotherapy (Roffe L et al., J Clin Oncol. 2004 Nov. 1; 22(21):4418-24; Perumal S S et al., Chem Biol Interact. 2005 Feb. 28; 152(1):49-58). The positive results are not always reported (Roffe et al., Journal of Clin Oncology 2004; 22(21) 4418-4424; Prieme H et al., Am J Clin Nutr. 1997 February; 65(2):503-7; Hodges et al., Biofactors 1999; 9(2-4):365-70; Lesperance et al., Breast Cancer Res Treat. 2002 November; 76(2):137-43) and the use of HMG-CoA inhibitors which lower endogenous production of cholesterol and CoQ10 do not appear to be a pre-determinant to cancer (Sacks et al., Reply letters to the editor JACC 1999 33 (3): 897-898). On the other hand, CoQ10 has generally been used for a broad spectrum of other disorders, such as end-stage heart failure (Berman M et al., Clin Cardiol. 2004 May; 27(5):295-9; Erman A, Ben-Gal T, Dvir D, Georghiou G P, Stamler A, Vered Y, Vidne B A, Aravot D), chronic heart failure (Mortensen S A Biofactors. 2003; 18(1-4):79-89), hypertension, hyperlipidemia, coronary artery disease (Sarter B. J Cardiovasc Nurs. 2002 July; 16(4):9-20), heart complications associated with use of statin drugs (Langsjoen P H and Langsjoen A M. Biofactors. 2003; 18(1-4):101-11; Chapidze G et al., Georgian Med News. 2005 January; (1):20-5), hypertriglyceridemia (Cicero A F et al., Biofactors. 2005; 23(1):7-14), chronic fatigue (Bentler S E et al., J Clin Psychiatry. 2005 May; 66(5):625-32), alzheimer's and parkinson's disease (Ono K et al., Biochem Biophys Res Commun. 2005 Apr. 29; 330(1):111-6; Beal M F. J Bioenerg Biomembr. 2004 August; 36(4):381-6), oxidative neurodegenerative injury (Somayajulu M et al., Neurobiol Dis. 2005 April; 18(3):618-27), migraine headaches (Sandor P S et al., Neurology. 2005 Feb. 22; 64(4):713-5), age-related loss of cognitive function (McDonald S R et al., Free Radic Biol Med. 2005 Mar. 15; 38(6):729-36), muscle and cardiomyopathies (Lalani S R et al., Arch Neurol. 2005 February; 62(2):317-20), hyperthyroidism (Menke T et al., Horm Res. 2004; 61(4):153-8), preeclampsia (Teran E et al., Free Radic Biol Med. 2003 Dec. 1; 35(11):1453-6) and cerebellar ataxia (Lamperti C et al., Neurology. 2003 Apr. 8; 60(7):1206-8).

CoQ10 has also been incorporated into a range of patented formulations such as those known to treat cancer (WO 02/078727, Feb. 24, 2004, Van De Wiel), endothelial dysfunction (CN1471390, Jan. 28, 2004, Watts and Playford), skin (US2005036976, Feb. 7, 2005, Rubin and Patel), cardiovascular and weight gain (US2004028668, Feb. 12, 2004, Gaetani), arteriosclerosis (US2004248992, Dec. 9, 2004, Fujii et al.,) periodontosis (U.S. Pat. No. 6,814,958, Nov. 9, 2004, Sekimoto), post-surgical ophthalmologic pathologies (U.S. Pat. No. 6,787,572, Sep. 7, 2004, Brancato, et al.), neurodegenerative disease, memory loss (U.S. Pat. No. 6,733,797, May 11, 2004, Summers), mitochondrial disorders (CA2285490, Apr. 7, 2001, Sole and Jeejeebhoy), diabetes (CA2476906, Sep. 25, 2003, Fujii et al,) and as a part of formulations that comprise antioxidants (CA2457762, Apr. 10, 2003, De Simone), hair or scalp treatment (CA 2444282, Dec. 19, 2002, Kawabe), sunscreen (CH693624, Nov. 28, 2003, Gecomwert) and food supplements (U.S. Pat. No. 6,642,277, Nov. 4, 2003, Howard et al.,).

DMBQ/and ubiquinone(s) (0-45)±ubiquinone (50) can be combined with vitamin B2 (riboflavin: 7,8-dimethyl-10-ribityl-isoalloxazine), its derivatives and pharmaceutically acceptable salts. Vitamin B2 is a precursor to flavin mononucleotide (FMN) and flavin adenine dinucleotide (FAD), both which play a critical role in the oxidative metabolism of glucose and fatty acids. Our results indicate that unlike glucose or niacin (which propel anaerobic glycolysis in cancer cells), riboflavin, FMN and FAD can augment mitochondrial complex I enzyme function (over 1000× fold increase) and exert pre-eminent control in causing a robust acceleration of mitochondrial cell respiration (O₂ utilization) in cancer cells (Mazzio and Soliman, Biochem Pharmacol. 67:1167-84, 2004). The effects of riboflavins on complex I and IV, adversely impede anaerobic (substrate level phosphorylation) glucose utilization in the very opposite fashion to the effect of mitochondrial toxins such as MPP+ (also targeting complex I and IV), which accelerate anaerobic glucose utilization and create an optimal metabolic state (Mazzio and Soliman, Brain Res. 2003 Feb. 7; 962(1-2):48-60). These findings suggest that riboflavin could contribute toward the forcing of aerobic oxidative metabolism, and play a role in suppressing tumor growth. Interestingly, to this day, the role for riboflavin and cancer remains ambiguous and unclear. A number of earlier studies consistently corroborated that a riboflavin deficiency or the use of riboflavin antagonists (eg. diethyl riboflavin) had anti-tumor effects, where dietary riboflavin supplementation resulted in the acceleration of tumor growth and metastasis (Nutr Rev. 1974 October; 32(10):308-10; Shapiro et al., Cancer Res. 1956 August; 16(7):575-80). However, these findings were not always consistent and riboflavin was consistently found to be protective against carcinogenesis by azo compounds (Rivlin, Cancer Res. 1973 September; 33(9):1977-86). In contrast, others suggest that a deficiency in riboflavin may be a risk factor for the development of cervical dysplasia and various other types of cancer, where high intake of riboflavin is protective against a variety of cancers in both animals and humans (Thurnham et al., Nutr Cancer. 1985; 7(3):131-43; Chen et al., Nutr Cancer. 2002; 42(1):33-40; Powers H J, Am J Clin Nutr. 2003 June; 77(6):1352-60; Petridou, Nutr Cancer. 2002; 44(1):16-22; La Vecchia et al., Int J Cancer. 1997 Nov. 14; 73(4):525-30; Key, Proc Nutr Soc. 1994 November; 53(3):605-14.). Our findings suggest its protective role may also be through its unique ability to propel oxidative respiratory function in cancer cells.

The use of riboflavin has been described in few patent publications regarding cancer. Of these, its use has been illustrated for the reduction of toxic effects of chemotherapy (WO03/045372, Jun. 5, 2003, Burzynski and Kammerer), in combination with lumichrome derivative for suppression of tumors (JP6279445, Oct. 4, 1994, TSuzaki), as an enrichment with vitamin E, to chinese medicines scorpion, Fructus lycii, Radix glycyrrhizae, Fructus zizyphi jujubae, Rhizoma smilacis glabrae, and Flos chrysanthemi and crop liqour for treatment of cancer and senility (CN1081467, Feb. 2, 1994, Belin) and as a component to anti-cancer foods with nicotinic acid and amino acids (JP58170463, Oct. 7, 1983, Asoujima). Moreover, riboflavin and its derivatives have been described in patent publications for a wide variety of other maladies including toxic shock (WO 97/36594, Mar. 28, 1997, Araki et al.,), infections, septic shock (WO 02/074313, Mar. 19, 2003, Araki et al.,), headache (WO 02/11731, Jul. 20, 2001, Valletta and Banchetti), high cholesterol (WO 02/34261, Oct. 21, 2001, Ohsawa et al.,) weight loss (WO 02/060278, Jun. 13, 2001, Gaetani and Cavattoni), acne (U.S. Pat. No. 6,558,656, Jun. 6, 2003, Mann), diseases of genital and mucous membranes (U.S. Pat. No. 6,020,333, Feb. 1, 2000, Berque), viral infections (CA 2174552, Apr. 27, 1995, Washington et al.,), macular degeneration (U.S. Pat. No. 5,075,116, Dec. 24, 1991, LaHaye), immune disorders (WO 03/084545, Apr. 9, 2003, Araki et al.,), hemorrhoids (CA 1147656, Jun. 7, 1983, Breskman), and as a part of nutritional supplement formulations as one of the B-complex vitamins (U.S. Pat. No. 6,245,360, Jun. 12, 2001, Markowitz).

The formulation at most should simultaneously augment mitochondrial oxidative respiration and inhibit LDH. LDH inhibition should be specifically targeted, where the remainder of the glycolytic pathway to the production of pyruvate remains unaffected. The reason for this is that the glycolytic pathway converts 1 mole of glucose to 2 moles of pyruvate, which then can diverge to fuel either anaerobic metabolism through LDH or it is transported to the mitochondria where it is converted to acetyl-CoA by pyruvate dehydrogenase to sustain aerobic (oxidative) metabolism. The latter metabolic pathway leads to the ultimate generation of reducing equivalents (NADH2/FADH2) by clockwise tricarboxylic acid cycle activity, for entry into the electron transport chain to produce ATP (Armstrong and Frank, Biochemistry-Second Edition, New York, Oxford University Press Inc., 1983). In cancer cells, the predominant fate of pyruvate is lactic acid through activity of LDH in the cytosolic compartment. And, to ensure little to no side effects—the formulation should not affect the remainder of the glycolytic pathway—which is a common ground between cancer and the host. While the substitution of any specific LDH inhibitor would be effective, we tested a range of polyphenolic compounds and herbal extracts for efficacy in inhibiting the LDH enzyme directly. Some flavonoids were not as potent as others. For example sesamol, apigenin, rutin and diosmin exhibited mild to no effect, where as 2′,3,4′5,7-pentahydroxyflavone (herein also referred to as “morin”), was fairly potent and effective. Prior art relating to the specific use of morin has described its use either alone or in combination with other flavonoids in patent publications for antimicrobial agents (JP2004250406. Sep. 9, 2004, Danno Genichi and Arima Hidetoshi), treatment of diaper rash (JP2004091338, Mar. 25, 2004, Tamura Kokichi), an anti-tumor agent (JP2001055330, Feb. 27, 2001, Tanaka Takuji), substances that control plant fertility (U.S. Pat. No. 5,733,759, Mar. 31, 1998, Taylor Loverine and Mo Yinyuan) and treatment of chlamydial infection (CA 2419716, Feb. 21, 2002, Vuorela, Pia et al.,) or radiation dermatitis (U.S. Pat. No. 6,753,325, Jun. 22, 2004, Rosenbloom). Moreover, research studies have demonstrated the efficacy of morin against proliferation of carcinoma cells through a mechanism thought to involve inactivation of the cell cycle kinase and activation of the mitogen/stress pathway kinases (Brown J, O'Prey J, Harrison P R., Carcinogenesis. 2003 February; 24(2):171-7) and inhibition of topoisomerase I (Boege F, Straub T, Kehr A, Boesenberg C, Christiansen K, Andersen A, Jakob F, Kohrle J., J Biol. Chem. 1996 Jan. 26; 271(4):2262-70). Pentaallyl ethers of morin are also known to be anti-tumor agents, which can inhibit p-glycoprotein ATP efflux of chemotherapy drugs in drug resistant cells (Ikegawa et al., Cancer Letters: 2002; 177: 89-93). Our studies suggest a possible role for morin in antagonizing the function of LDH.

In addition, we screened a large number of herbal alcohol extracts for LDH inhibition. While many herbal compounds such as fenugreek, ginseng, dill, anise, cardamom, peppermint, chamomile, basil and cilantro were not effective, ethanol extracts of rosemary (Rosmarinus officinalis) and myrrh (Commiphora myrrha) were cytotoxic and effective in providing LDH inhibitory effects. While there are meager patent publications that describe the use of rosemary for the treatment of cancer, experimental research corroborates its capacity to antagonize tumor growth. For example carnosol, a phenolic compound extracted from rosemary, is toxic against acute lymphoblastic leukemia cells (Dorrie J, Sapala K, Zunino S J, Cancer Lett. 2001 Sep. 10; 170(1):33-9), human epithelial cell lines (Mace K, Offord E A, Harris C C, Pfeifer A M. Arch Toxicol Suppl. 1998; 20:227-36) and against colon cancer in vivo (Moran A E, Carothers A M, Weyant M J, Redston M, Bertagnolli M M. Cancer Res. 2005 Feb. 1; 65(3):1097-104). The extract of rosemary can increase the sensitivity and prevent the efflux of chemotherapeutic agents in drug resistant MCF-7 human breast cancer cells (Plouzek C A, Ciolino H P, Clarke R, Yeh G C., Eur J Cancer. 1999 October; 35(10):1541-5) and can inhibit 7,12-dimethylbenz[a]anthracene induced mammary tumorigenesis in female rats (Singletary K, MacDonald C, Wallig M. Cancer Lett. 1996 Jun. 24; 104(1):43-8). The use of rosemary has been used in formulations described in patent publications that demonstrate a range of products such as antimicrobial agents (U.S. Pat. No. 6,846,498, Jan. 25, 2005, DeAth, et al.), an aid to quit smoking (U.S. Pat. No. 6,845,777, Jan. 25, 2005, Pera), antioxidant sunscreen (U.S. Pat. No. 6,831,191, Dec. 14, 2004, Chaudhuri), nutritional supplement formulations (U.S. Pat. No. 6,827,945, Dec. 7, 2004, Rosenbloom), foods and personal care products (U.S. Pat. No. 6,844,020, Jan. 18, 2005, Johnson et al.,) and for the treatment of skin disease (U.S. Pat. No. 6,800,292, Nov. 5, 2004, Murad), diabetes (U.S. Pat. No. 6,780,440, Aug. 24, 2004, Naguib), allergies (U.S. Pat. No. 6,811,796, Nov. 2, 2004, Yoshida), ulcers (U.S. Pat. No. 6,638,523, Oct. 28, 2003, Miyazaki, et al.), inflammatory disorders (U.S. Pat. No. 6,541,045, Apr. 1, 2003, Charters, et al.), pain (U.S. Pat. No. 6,444,238, Sep. 3, 2002, Weise) and psoriasis (U.S. Pat. No. 6,403,654, Jun. 11, 2002, De Oliveira).

Myrrh can also be used for/or adjunct to the LDH inhibitor component. Myrrh is primarily known for its anti-parastic (Massoud A M, El Ebiary F H, Abou-Gamra M M, Mohamed G F, Shaker S M., J Egypt Soc Parasitol. 2004 December; 34(3 Suppl):1051-76; Soliman O E, El-Arman M, Abdul-Samie E R, El-Nemr H I, Massoud A. J Egypt Soc Parasitol. 2004 December; 34(3):941-66) anti-microbial (El Ashry E S, Rashed N, Salama O M, Saleh A. Pharmazie. 2003 March; 58(3):163-8) and anti-fungal properties (Dolara P, Corte B, Ghelardini C, Pugliese A M, Cerbai E, Menichetti S, Lo Nostro A Planta Med. 2000 May; 66(4):356-8). And, although there is research investigating its use against infections, there is little to no experimental research describing its use in the treatment of cancer. On the other hand, a number of patent publications have described the use of myrrh in complex chinese medicinal herbal formulations to treat cancer (WO 00/50053, Feb. 29, 1999, Sofer et al., US2004219226, Nov. 4, 2004, Lee et al.; CN1413659, Apr. 30, 2003, Changkui; CN1448164, Oct. 15, 2003, Wenxiu; CN1302622, Jul. 11, 2001, Fangy; CN1237447, Dec. 8, 1999, Shaoxian; CN1203801, Jan. 6, 1999, Ruifen and Fengxiang; U.S. Pat. No. 5,876,728, Mar. 2, 1999, Kass et al.; CN1151293, Jun. 11, 1997, Fawang and Qing; CN1133725, Oct. 23, 1996, Tang; CN1107351, Aug. 30, 1995, Fang et al., CN1061908, Jun. 17, 1992, Yang et al.,; CN1058911, Feb. 26, 1992, Chen). Moreover, the use of myrrh has been described in patent publications describing a range of products such as appetite suppressant toothpaste (CA2485562, Jun. 10, 2004, Zuckerman), deodorant (JP2004160216. Jun. 10, 2004, Imanaka), burn ointment (CN1440775, Sep. 10, 2006, Xiao et al.,), arthritis medication (CN1438001, Aug. 27, 2003, Chen), massage oil (CN1449785, Oct. 22, 2003, Li), anti-viral agents (WO 99/38522, Jan. 29, 1999, Preus and Dzieglewsak), animal foods (U.S. Pat. No. 6,652,892, Nov. 25, 2003, McGenity, et al.) and soaps (U.S. Pat. No. 6,680,285, Jan. 20, 2004) to name a few.

Black walnut (Juglans Nigra) extract was also found to be a potent LDH inhibitor. However, it inherently contains compounds such as 5-hydroxy 1,4-napthoquinone, which upon further analysis were found to inhibit pyruvate kinase. The inhibition of pyruvate kinase could render potential toxic side effects to the host. Therefore, future research would be required to identify chemicals in blackwalnut extract that could specifically inhibit LDH, without altering the remainder of the glycolytic pathway. Although there is little documentation in either research or patented literature, historical herbal literature indicates that black walnut is known to treat intestinal parasites, worms, warts, growths, eczema, psoriasis, lupus, herpes, and skin parasites.

Herbal substances such as rosemary and myrrh are comprised of polyphenolic compounds that have intrinsic anti-oxidant, antimicrobial and anti-inflammatory properties (Theoharides T C, Alexandrakis M, Kempuraj D, Lytinas M. Int J Immunopathol Pharmacol. 2001 September; 14(3):119-127; Makris D P, Rossiter J T. J Agric Food Chem. 2001 July; 49(7):3370-7; Aggarwal B B, Shishodia S. Ann N Y Acad. Sci. 2004 December; 1030:434-41; Lai P K, Roy J. Curr Med. Chem. 2004 June; 11(11):1451-60). For this reason, polyphenolic compounds, herbs and spices are known to have robust therapeutic value against a large range of inflammatory disorders such as diabetes, allergies, cardiovascular disease, infections, retinopathy, septic shock, neurodegenerative disorders, liver disease, cataracts, periodontal disease and arthritis, which have been described in a plethora of research publications (Alt Med Rev 1996; 1(2):103-111). Moreover, a study examining 50 patented polyphenolic plant derived drugs also describes formulations that contain over 685 species of plants in defined treatments for inflammatory disorders such as arthritis, rheumatism, acne skin allergies and more (Darshan S, Doreswamy R., Phytother Res. 2004 May; 18(5):343-57), indicating the broad spectrum of utility for this class of compounds for the treatment of diseases other than cancer (Arts and Hollman, Am J Clin Nutr. 2005 January; 81(1 Suppl):317S-325S). In summary, this invention entails a holistic method for preventing and treating cancer by using a specific combination of chemicals or agents that target specific means in order to switch the body's metabolism to an aerobic state, thereby specifically blocking glucose metabolism in the tumor.

### BRIEF SUMMARY OF INVENTION

The present invention is directed to a composition and method of treating cancer. The embodiment discloses a natural pharmaceutical formulation designed to exploit the vulnerability of cancer tissue with regards to its anaerobic requirement for non-oxidative phosphorylation of glucose to derive energy, which is in direct opposition to the host. The formulation is intended to target central pathways that should block the use of glucose for production of ATP only in cancer cells. More particularly, the composition of this invention is comprised of a combination of one or more of the following (A) 2,3-dimethoxy-5-methyl-1,4-benzoquinone, ubiquinones (5-45) (B) compound(s) or substance(s) capable of augmenting oxidative phosphorylation such as a riboflavin containing compound and/or ubiquinone (50) (C) 2′,3,4′5,7-pentahydroxyflavone or an analogous alternative capable of inhibiting lactic acid dehydrogenase and (D) any chemical(s) or substance(s) that are capable of inhibiting one or more of acetate-coA ligase, malate synthase, isocitrate lyase, aconitase, phosphoenolpyruvate carboxylase/carboxykinase, glycolate oxidase, phosphoglycolate phosphatase, glycolaldehyde dehydrogenase, pyruvate carboxylase, citrate lyase, ferridoxin oxidoreductase, fructose 1,6-bisphosphatase, 2,3-diphosphoglycerate mutase, propionyl CoA carboxylase, malic enzyme, acetyl CoA carboxylase and ribulose-1,5-bisphosphate carboxylase. The combination of these compounds can be strategically used to favor oxidative decarboxylation reactions, suppress the conversion of non-glucose carbon based compounds through gluconeogenesis and initiate collapse of glycolysis in tumor tissue, a chemical manipulation that should be non-toxic or perhaps even beneficial to normal respiring host tissue. A preliminary formulation has been pilot tested, demonstrating anti-tumor activity and efficacy comparable to tamoxifen (in vitro) and taxol® (in vivo), showing no adverse effects on the animal as determined by weight, behavior, food/water intake or excretory function.

### BRIEF DESCRIPTION OF THE DRAWINGS

FIG. 1 Summary of results—Re: Mazzio and Soliman, Biochem Pharmacol. 67:1167-84, 2004. The following schematic is a brief description summarizing the findings in this study. Briefly, the paper describes the function of ubiquinone (50) in augmenting the kinetic activity of mitochondrial complex II in cancer cells, while having no positive effect on mitochondrial respiration. On the other hand, riboflavin appears to control the kinetic activity of complex I, which drastically potentiates the rate of mitochondrial oxygen consumption through complex IV. These findings describe the inverse relationship between a reduction in mitochondrial oxygen consumption (ie. mitochondrial poisons), and anaerobic ambient conditions that foster enhanced glycolytic activity, leading to metabolic activation, glucose depletion and cell death by starvation. Conversely, a rise in ambient oxygen concentration or enhanced mitochondrial oxygen consumption (riboflavin) appears to disrupt glycolysis or glucose utilization. Further, neuroblastoma cells have the ability to thrive under completely anaerobic conditions (ie. in the presence of mitochondrial poisons such as MPP+, in the absence of O₂, or the removal of dissolved O₂ with dithionite) given that glucose supply is sustained. Briefly, our findings indicate that the mode of MPP+ toxicity in a blastoma cell model for the study of Parkinson's disease occurs through the propelling of anaerobic glucose metabolism leading to subsequent depletion of glucose supply and cell death by starvation. These findings may be relevant to the study of cancer as they demonstrate the dependency of malignant cells to derive ATP solely through substrate level phosphorylation and the adverse effects of optimized mitochondrial function on anaerobic glycolysis.

FIG. 2—The toxicity of selected plant extracts and 2′,3,4′5,7-pentahydroxyflavone were previously determined on N-2A neuroblastoma cells prior to examination of their effects on kinetic activity of pyruvate kinase (PK) and LDH (data not shown). Briefly, the effects of experimental compounds on PK and LDH Type V, resembling that inherent to human cancer (Koukourakis et al., Br J Cancer. 2003; 89:877-85; Augoff and Grabowski. Pol Merkuriusz Lek 2004; 17:644-7; Nagai et al., Int J Cancer. 198815;:10-6; Evans et al., Biol. Chem. 1985; 260:306-14) were determined in pure isolated enzyme preparations. Briefly, PK Type III (from rabbit muscle [2.7.1.40]) was prepared in distilled water+HEPES (pH 7.5), at a concentration of 0.5 enzyme U/ml. Pyruvic acid was converted to lactate in the presence of LDH (from rabbit muscle, type V-S [EC 1.1.1.27]), at a concentration of 10 U/ml in the presence of adenosine, 2′,5′-diphospahte (ADP) (1.5 mM), 3-NADH (1 mM)±magnesium sulfate (MgSO₄) (5 mM). Experimental compounds were incubated with the enzyme solution for 10 minutes and addition of 1 mM phosphoenolpyruvate (PEP) prepared in distilled water started the reaction. Negative controls were established for all compounds tested. Enzyme activities were determined by spectrophotometric analysis using a UV spectrometer at 340 nm, by monitoring the oxidation of NADH. Experimental compounds that blocked the reaction through the PK/LDH cascade, were re-analyzed for LDH inhibition. LDH activity was achieved using an enzyme reaction mixture, minus PK or PEP, and starting the reaction with pyruvate (1 mM) prepared in buffered distilled water. Validation studies for LDH kinetic activity were established by monitoring the oxidation of NADH over time and concentration with dual detection quantifying lactic acid using a lactate oxidase based calorimetric enzymatic assay (Procedure No 735, Sigma Diagnostics, St. Louis, Mo.). FIG. 2A describes the effect of tumoricial plant extracts and 2′,3,4′5,7-pentahydroxyflavone on inhibition of PK/LDH activity. The data represent reaction rate of NADH oxidation in the presence of enzyme/cofactor reagents+PEP (1 mM)±varying concentration of experimental compounds at 30 Min. The data are expressed as the Mean±S.E.M., (n=4). Significance of difference from the controls were determined by a one-way ANOVA, followed by a Tukey mean comparison post hoc test, [*] group P<0.001, *P<0.001. FIG. 2B describes the effect of tumoricial plant-extracts and 2′,3,4′5,7-pentahydroxyflavone on inhibition of LDH activity. The data represent reaction rate of NADH oxidation in the presence of enzyme/cofactor reagents+pyruvate (1 mM)±single level of experimental compound over time. The data are expressed as the Mean±S.E.M., (n=4). Significance of difference from the control was determined by a two-way ANOVA, [*] P<0.001.

FIG. 3A describes the evaluation of 3-bromopyruvate (3-BP) versus 2,3-dimethoxy-5-methyl-1,4-benzoquinone (2,3-DMBQ) on growth inhibition of MCF-7 mammary carcinoma cells. Briefly, cells were grown in Eagles MEM medium with 20 mg insulin/ml and 10% calf serum and plated at 5′ 104 cells in 24 well plates. Appropriate positive (tamoxifen) and negative (no drug) controls were maintained simultaneously. After 24 hour incubation, cells were trypsined and collected by centrifugation, re-suspended in fresh media and cells were counted using trypan blue dye on a hemacytometer. The data are expressed as the mean±S.E.M., n=3, and the significance of difference from the controls was determined by a one way ANOVA, followed by a Tukey mean comparison post hoc test (*=P<0.001). FIG. 3B represents the evaluation of 3-BP versus 2,3-DMBQ on cell viability in N2A neuroblastoma cell line. Briefly, the experimental media consisted of DMEM (without phenol red), supplemented with 1.8% FBS (v/v), penicillin (100 U/ml)/streptomycin (0.1 mg/ml), 4 mM L-glutamine and 20 μM sodium pyruvate. Cells were plated at approximately 0.5×106 cells/ml in 96 well plates. A stock solution of each experimental compound was prepared in HBSS containing 5 mM HEPES, adjusted to a pH of 7.4. After 24 hours incubation at 37° C. and 5% CO₂/atmosphere almar blue indicator dye was used to assess cell viability. Quantitative analysis of dye conversion was measured on a microplate fluorometer—Model 7620-version 5.02, Cambridge Technologies Inc. with settings fixed at [550/580], [excitation/emission], wavelengths. The data are expressed as the mean±S.E.M., n=4, and the significance of difference from the controls was determined by a one way ANOVA, followed by a Tukey mean comparison post hoc test (*=P<0.001).

FIG. 4A describes the effect of a natural pharmaceutical formulation (NPF) on MD-MB-231 mammary carcinoma in Nu/Nu female mice. Briefly, 6 week old female Nu/Nu mice were kept in an autoclaved micro isolator cage, maintained under pathogen free conditions. The tumors were ascetically surgically removed and transferred to a sterile Petri dish containing RPMI-1640. The homogenate was centrifuged, pelleted, resuspended into a concentration of 10 million cells/ml and injected into the mammary fat pad. The tumors were established by day 9 after implant and treatment began. The formula was prepared in sterile saline, and administered by i.p. injection for 3 days and s.c for the next 3 days, stopping at day 15. Taxol—(24 mg/kg in 2% PEG 300, 8% cremophor CL an 80% sterile Saline) was administered i.v. Intermittently up to day 19 (days 10, 13, 16 and 19). Since there were no signs of toxicity with the formulation, to gain greater understanding as to the effects of this drug, the dose was increased to 1.5× and 2× for two regimens implemented at day 35, and 37 of the tumor implantation. The data represents tumor volume estimation (mm3) and expressed as the mean±S.E.M., n=4, for treatment groups, with n=1 for the control. FIG. 4B describes the effect of a NPF treatment on weightloss, behavior and health. There were no deaths reported in the experiment due to toxicity. The control animals had a moderate weight gain within the acceptable limits for normal growth. There was a loss of weight with in the taxol treatment group. In a comparison chart, animals treated with the formulation showed no weight loss. The formulation was well tolerated by the animals in the dosing regimen, and showed no signs of toxicity. The food and water intake was normal and the same for the excretory functions. The animals showed no other behavioral changes. The data represents weight gain (g) and are expressed as the mean±S.E.M., n=4, for treatment groups, with n=1 for the control. Treatment period (!---!)

### DETAILED DESCRIPTION OF THE PREFERRED EMBODIMENT

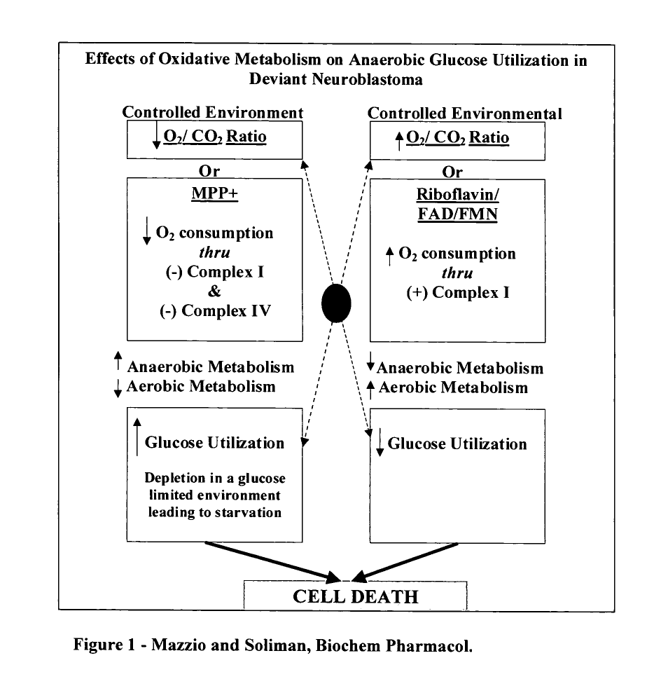

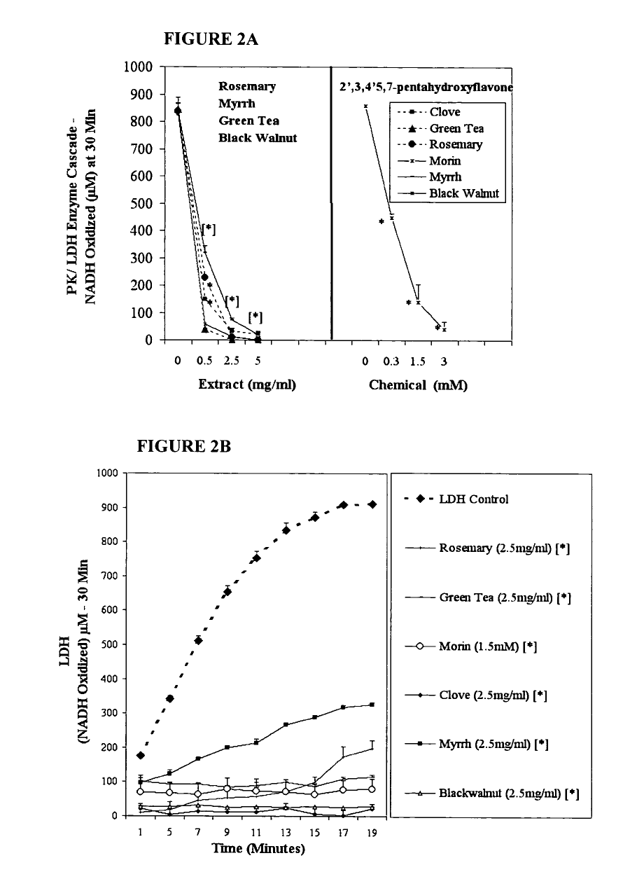

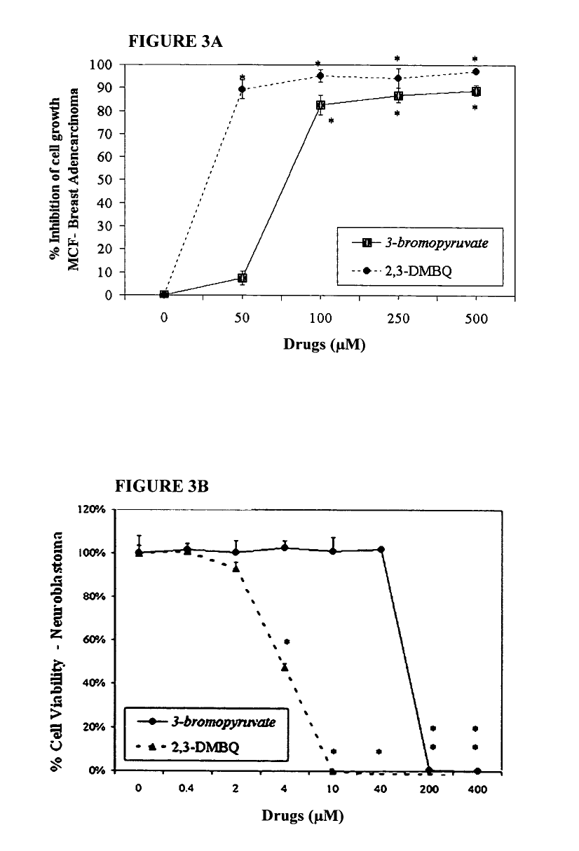

The embodiment of the present invention relates to a holistic chemotherapy agent for treatment of cancer in humans and animals. As summarized from the background literature and experiments as described above, the formulation attempts to shift the body toward a more aerobic state, which should be lethal to cancer but beneficial to the host. Briefly, 2,3-dimethoxy-5-methyl-1,4-benzoquinone (ubiquinone (0)) herein termed “DMBQ” and the short chain ubiquinones appear to adversely target a predominant cytosolic anaerobic metabolic pathway involving the conversion of non-glucose carbon based substrates into glucose. Ubiquinone (50) plays an important role in oxidative phosphorylation where it shuffles electrons to flavoprotein enzymes (requiring FMN prosthetic groups) and cytochromes, and translocates protons to generate a proton-motive force by which to propel oxidative phosphorylation and aerobic production of ATP. Riboflavin, FAD and FMN play a paramount role in electron transport, the function of ubiquinone oxidoreductases, the facilitation of aerobic metabolism of glucose, and can increase O₂ utilization by the mitochondria in cancer cells in excess of 400%, all which correspond to the impedance of anaerobic glycolysis in cancer cells (Mazzio and Soliman, Biochem Pharmacol. 67:1167-84, 2004; publication summary of FIG. 1. Further, we have evaluated a number of compounds that are capable of inhibiting LDH-5 (the isoform most resembling that inherent to human) (Koukourakis et al., Br J Cancer. 2003; 89:877-85; Augoff and Grabowski. Pol Merkuriusz Lek 2004; 17:644-7; Nagai et al., Int J Cancer. 198815;:10-6; Evans et al., Biol. Chem. 1985; 260:306-14), some of which are displayed in FIG. 2. Moreover, individual components of the formulation were tested in vitro at Florida A & M University using neuroblastoma models from various species, and at the University of Miami, using MCF-7 cell line derived from the pleural effusion of a female patient with metastatic breast carcinoma (FIGS. 3A,B). Both report similar effects on all compounds tested (data not shown) and the effects of DMBQ were >50× more toxic than bromopyruvate, which is currently considered a cancer breakthrough due to its lethal effects on certain types of tumors, with little observable toxic effects to the host (BBC News, Jul. 16, 2002). While we only show the data for DMBQ, all compounds had tumoricidal properties, and additive effects of LDH inhibitors and flavin derivatives lowered the LC₅₀ of DMBQ. Additionally, preliminary studies in our lab indicated that 3-bromopyruvate was not an inhibitor of LDH-5 at any concentration where the compound exhibited tumoricidal properties, suggesting its effects are through an unknown mechanism, independent of LDH. Oddly, other known LDH-5 inhibitors such as oxamate were ineffective in inhibiting LDH or inducing toxicity suggesting that much more research will be required to synthesize and evaluate specific LDH inhibitors as anti-cancer agents. The combination of the substances that comprise this invention, should augment the concentration of pyruvate and its utilization for aerobic mitochondrial cell respiration in normal cells, while blocking anaerobic energy production from glucose in cancer cells. In brief overview, while there could be many mechanisms involved with observed efficacy, it is the intent that the formulation shift the metabolism of the host toward a more aerobic state thereby specifically blocking ATP production in the tumor.

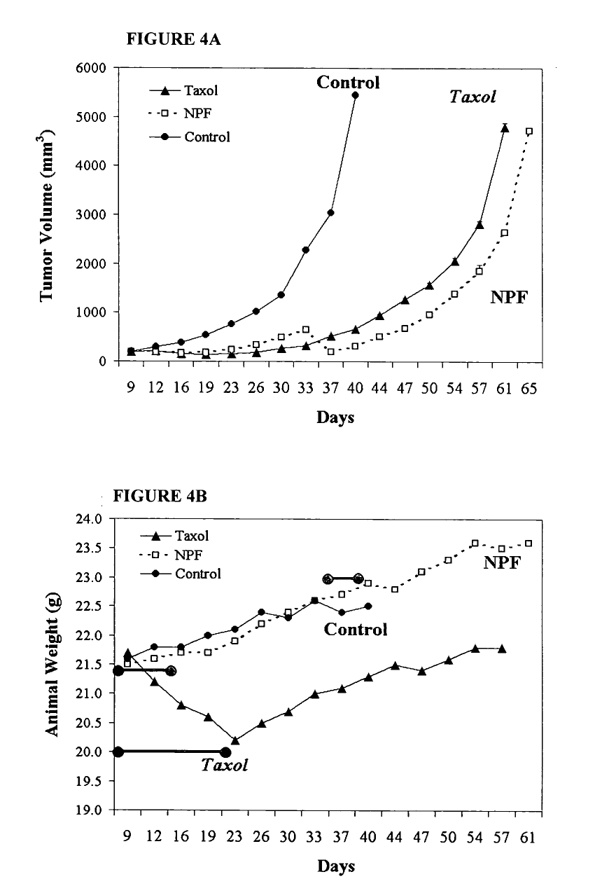

A working example comprised of a tri-fold formulation containing an active ingredient from each of the primary classified components as listed above, was analyzed for efficacy against a tumor model in mice. These data present specifically the efficacy, utility and substantial effect of a test formulation which consisted of riboflavin, 2,3-dimethoxy-5-methyl-1,4-benzoquinone and 2,3,4,5,7-pentahydroxyflavone. The preliminary formulation was submitted to Kard Scientific (Boston, Mass.) for a small pilot study to determine efficacy against MD-MB-231 human mammary carcinoma in a xenograft model using Nu/Nu nude mice (FIGS. 4A,B). In this study, two treatment groups were established and consisted of the formulation and taxol®, both compared to a non-treated control. Both taxol® and the formulation showed a reduced tumor growth and growth latency in comparison to a vehicle control. Unlike taxol®, where there was weight loss observed during treatment, administration of the formulation accompanied no sign of toxicity, behavioral changes or weight loss in test animals. The formulation was well tolerated, where food and water intake, behavior and excretory functions were maintained at a normal level. The animals showed no other behavioral changes. The route of administration in this study was s.c. and i.p, indicating the formulation would be powerful if administered iv, like taxol. Further, the formulation is effective in its water-soluble form, yet readily modifiable to suit a large range of solubilities based on the number of side chain units associated with the quinoid base. This fulfills a current need to establish treatment that does not require emulsifying agents or solubilizing vehicles (ie cremaphor®), which can lead to further complications such as hypersensitivity reactions.

More definitively, the active component(s) of the formula are comprised of a combination of one or more of the following: A) 2,3-dimethoxy-5-methyl-1,4-benzoquinone, ubiquinones (5-45), their corresponding analogues, derivatives or prodrugs B) any chemical (s), substance(s) or agent(s) capable of augmenting mitochondrial oxidative phosphorylation herein termed “OXPHOS (+)”, such as riboflavin (vitamin B2) and its derivatives, flavin adenine dinucleotide, flavin mononucleotide or analogs and/or ubiquinone (50) and C) 2,3,4,5,7-pentahydroxyflavone or a suitable alternative such as chemicals(s), substances (s), agent(s) or extract(s) capable of inhibiting LDH, herein termed “LDH (−)”. The term OXPHOS (+) is further defined as any chemical(s), substance(s) or agent(s) that can augment or contribute to the function of NADH:ubiquinone oxidoreductase (complex I), succinate dehydrogenase-CoQ oxoreductase (complex II), ubiquinol:cytochrome c oxidoreductase (complex III), cytochrome c oxidase (complex IV), ATP synthase (complex V), the Krebs cycle and mitochondrial respiration either directly or indirectly. These include metabolic precursors or compounds required for the biosynthesis of coenzyme Q10, Krebs cycle or respiratory enzymes or the function thereof. For example constituents required for decarboxylation reactions/pyruvate dehydrogenase activity such as thiamin, biotin, pantothenate or lipoic acid, constituents required for ubiquinone synthesis such as tyrosine, tetrahydrobiopterin (THB), vitamins B2, B6, B12, folate, niacin, vitamin C, pantothenic acid (Folkers et al., Biochem Biophys Res Commun 1996 244: 358-363) and ubiquinone metabolic precursors including para-hydroxybenzoate, para-hydroxycinnamate, para-hydroxyphenylpyruvate, para-hydroxyphenyllactate, polyprenyl-para-hydroxybenzoate, tyrosine, phenylalanine and isopentyl-diphosphate. The determination of compounds to be included in the OXPHOS (+) component, can be assessed by effects on the function of mitochondria/enzymes derived from any relevant source including but not limited to bacteria, animal, plant, yeast, mold or tumor. The term LDH (−) is further defined as any compound(s), chemical(s) or agent(s) that can inhibit preferably LDH-5, the LDH inherent to cancer, as well as any other pertinent isoforms that can be used experimentally and relate to the LDH in cancer, including that derived from any source including but not limited to plant, bacteria, yeast, mold, fungus, animal or tumor. The LDH (−) component should be capable of inhibiting the LDH enzyme inherent to cancer or LDH-5 also termed “LDH-V”, at concentrations that juxtapose tumoricidal effects, indicating the mechanism of action involves inhibition of LDH. Additionally, a further component herein termed anaerobic inhibiting component “AIC (−)” can also be incorporated into the invention, being defined as compounds(s) or substance(s) other than DMBQ, that aid in blocking the conversion of carbon-2 intermediates into energy, CO₂ into carbon intermediates or inhibit enzymes that utilize CO₂ as a substrate/cofactor. The AIC (−) component is further defined as any agent(s), chemical(s) or substance(s) that are capable of inhibiting anaplerotic carboxylase enzymes, the glyoxylate shunt, reductive tricarboxylic acid cyle, the calvin-benson cyle or gluconeogenesis either indirectly or directly, and more specifically, inhibit one or more of the following enzymes: acetate-coA ligase, malate synthase, isocitrate lyase, aconitase, phosphoenolpyruvate carboxylase/carboxykinase, glycolate oxidase, phosphoglycolate phosphatase, glycolaldehyde dehydrogenase, pyruvate carboxylase, citrate lyase, ferridoxin oxidoreductase, fructose 1,6-bisphosphatase, propionyl CoA carboxylase, malic enzyme, acetyl CoA carboxylase, 2,3-diphosphoglycerate mutase, and ribulose-1,5-bisphosphate carboxylase.

Even more definitively, DMBQ, other ubiquinones or ubiquinone (50) in the OXPHOS (+) component, can include their corresponding hydroquinones, ubichromenols, ubichromanols or synthesized/natural derivatives. Benzoquinones of this family are properly referred to as either “Coenzyme Qn” where n designates the number of isoprene units (also termed “prenyl”) in the isoprenoid side chain, or alternatively, “ubiquinone (x)” where x designates the total number of carbon atoms in the side chain. The quinones of the coenzyme Q series differ in chemical structure and form a group of related, 2-3-dimethoxy-5-methyl-benzoquinones with variation in length of the polyisoprene side chain. The term “ubiquinone” is represented by the following base structure: 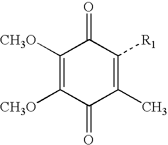
wherein R₁ is equal to or greater than 0 isoprene (3-methyl-2-butenyl) unit (s)

For example ubiquinone (5), which corresponds to the structure: 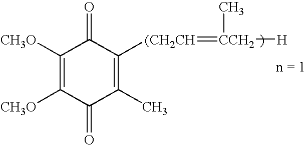
wherein n is equal to the number of isoprene units

In structure, coenzyme Q resembles vitamin K (base nucleus: 2-methylnaphthoquinone), the plastoquinones (base nucleus: 2,3-dimethylbenzoquinone), tocopherolquinones (base nucleus: 2,3,5-trimethylbenzoquinone) and menoquinone (base nucleus: 2-methyl-4-naphthoquinone) in that it possesses a quinone ring nucleus attached to a hydrocarbon tail (IUPAC definitions—Eur. J. Biochem. 1975 53: 15-18). The ubiquinone component (being that present in OXPHOS (+) component and/or ubiquinones (0-45)), may also be incorporated with or substituted by plastoquinones or vitamin E/K quinones. Ubiquinones can further include any oxidized or reduced (ubiquinol) forms such as CoQ0, ubiquinone (O), ubiquinol/ubichromenol (O), CoQ1, ubiquinone (5), ubiquinol/ubichromenol (5), CoQ2, ubiquinone (10), ubiquinol/ubichromenol (10), CoQ3, ubiquinone (15), ubiquinol/ubichromenol (15), CoQ4, ubiquinone (20), ubiquinol/ubichromenol (20), CoQ5, ubiquinone (25), ubiquinol/ubichromenol (25), CoQ6, ubiquinone (30), ubiquinol/ubichromenol (30), CoQ7, ubiquinone (35), ubiquinol/ubichromenol (35), CoQ8, ubiquinone (40), ubiquinol/ubichromenol (40), CoQ9, ubiquinone (45), ubiquinol/ubichromenol (45), CoQ10, ubiquinone (50), ubiquinol/ubichromenol (50) or any other derivative, analog, intermediate, precursor or pro-drug to these molecules.

The present invention can include ubiquinones (0+) derivatives, analogues, intermediates, precursors and prodrugs. Examples include rearrangements, modification, substitutions of the methyl, methoxy or carbonyl groups or the isoprenoid side chain with substituents such as alkyl groups including branched, cyclic and straight chain, alkylene, alkoxy, alkenyl, alkaryl, alkynyl, acyl, acylamino, acyloxy, cycloalkyl, cycloalkenyl, haloalkyl, aryl substituents including phenyl, napthyl and substituted phenyl substituents; aralkyl substituents including benzyl and tolyl substituents; halogen substituents including fluoro, bromo, chloro substituents; oxygen substituents including hydroxy, lower alkoxy, ether, carboxyl and ester substituents; nitrogen substituents including nitrogen heterocycles, heteroaryls, amides, amines and nitriles; sulfur substituents including thiol, thioether, thioalkoxy, thioaryloxy and thioesters and aldehydes, ketones and aromatic hydrocarbons or hydrogen. In addition to altering the methyl, carbonyl group and/or the methoxy groups with the above noted substituents, addition, rearrangment, replacement or modification of substituents also provides ubiquinones that are also included within the scope of this invention. Accordingly, small changes resulting from modification of the substituents or benzoquinone nucleus for any improved functionality are included within the scope of the present invention. Ubiquinones utilized in the present invention may be isolated in nature or synthetically produced using any method including known to one skilled in the art, by way of example (Weinstock et al., Journal of Chem Eng Data 1967 12(1) 154-155; Sato et al, Chem. Abst. 78:471, 1993; U.S. Pat. No. 5,254,590, Oct. 19, 1993, Malen et al; JP57021332, Feb. 4, 1982, Kiso Yoshihis; U.S. Pat. No. 6,225,097, May 1, 2001, Obata et al; U.S. Pat. No. 6,103,488, Aug. 15, 2000, Matsuda et al.; WO03/056024 Dec. 27, 2002 Yajima, K; JP57021332, Feb. 4, 1982 Kiso Yoshihisa; DE3221506 Dec. 8, 1983, Doetz Karl Heinz; U.S. Pat. No. 6,545,184, Apr. 8, 2003 Bruce Lipshutz and Paul Mollard; EP1354957, Oct. 22, 2003, Matsuda Hideyuki et al; JP55159797, Dec. 12, 1980, Hasegawa Yasuhiro), all of which could be incorporated by reference. One of ordinary skill in the art will appreciate that changes may be made to the ubiquinone structure for improved functionality to form a derivative without taking away from the tumoricidal function thereof. In embodiments of the present invention, ubiquinones (0-45)±the OXHPHOS (+) ubiquinone (50) can comprise from about 0 to about 100 weight percent (herein referred to as “wt %”) based on the total weight of the invention composition. More particularly, ubiquinones could be present in an amount of from about 30 to about 80 wt %, and more specifically in an amount of about 54 wt

The formulation can further include in its OXPHOS (+) component, a riboflavin containing compound, such as riboflavin, its pharmaceutically acceptable salts and derivatives: flavin mononucleotide (FMN), flavin adenine dinucleotide (FMN) or any other synthesized or natural derivative. In embodiments, the present invention includes a riboflavin containing compound or OXHPHOS (+) in an amount of from about 0 to about 100 wt % of the invention composition. More particularly, riboflavin or OXHPHOS (+) can be present in an amount of from about 15 to about 35 wt %, and more specifically in an amount of about 33 wt %. A riboflavin containing compound can also include compounds represented by the following base structure including its derivatives, intermediates, analogs, precursors and prodrugs including but not limited to 5-amino-6-(5′-phosphoribitylamino)uracil, 6,7-Dimethyl-8-(1-D-ribityl)lumazine, ribitol, lumichrome and 5,6-dimethylbenzimidazole. The term riboflavin is represented by the following base structure: 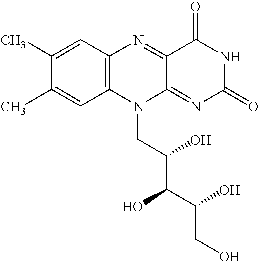
where in the isoalloxazine ring system of riboflavin contains methyl groups at C⁷ and C⁸ and a ribityl group at N¹⁰.

Examples of riboflavin derivatives can include rearrangements, modifications, substitutions of the methyl, carbonyl, amino or ribityl group groups with additional substituents such as such as alkyl groups including branched, cyclic and straight chain, alkylene, alkoxy, alkenyl, alkaryl, alkynyl, acyl, acylamino, acyloxy, cycloalkyl, cycloalkenyl, haloalkyl, aryl substituents including phenyl, napthyl and substituted phenyl substituents; aralkyl substituents including benzyl and tolyl substituents; halogen substituents including fluoro, bromo, chloro substituents; oxygen substituents including hydroxy, lower alkoxy, ether, carboxyl and ester substituents; nitrogen substituents including nitrogen heterocycles, heteroaryls, amides, amines and nitriles; sulfur substituents including thiol, thioether, thioalkoxy, thioaryloxy and thioesters and aldehydes, ketones and aromatic hydrocarbons. Accordingly, small changes resulting from addition, modification, rearrangment or replacement of the substituents or base structure are included within the scope of the present invention. Further, while it is doubtful that riboflavin and ubiquinone (50) alone could effectively place cancer in remission without adjunct chemotherapy or DMBQ, the use of a natural or synthetic potent AIC (−) or LDH (−) may in and of itself be effective. For that reason, the metes and bounds of the invention and claims delineate the AIC (−) or LDH (−) to be from 0-100% wt of total composition and can exist in combination with an OXPHOS (+) component (ie. riboflavin and ubiquinone (50))±DMBQ, which can vary to maximize efficacy.

In embodiments, an LDH (−) component can be present in an amount from about 0 to about 100 wt % of the total composition. More particularly, if combined with ubiquinones+a riboflavin containing compound, the LDH (−) can be present in an amount of from about 10 to about 50 wt %, and even more specific in an amount of about 13 wt %. It is important to mention that future research will be required to delineate maximum efficacy of therapeutic combinations and ranges. And, although our in vivo animal study incorporated the LDH (−) at 13%, we have further established evidence of the importance of this component, and its concentration above 75-80% may prove valuable. The LDH (−) can be morin (2,4,5,7-pentahydroxyflavone), which corresponds to the following structure and includes its derivatives, analogues and pro-drugs: 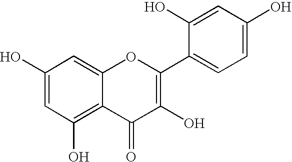
The LDH (−) may also be any chemical, polyphenolic or plant extract capable of inhibiting preferably LDH-5, any isoform of LDH inherent to cancer tissue, or any other relevant isoform of LDH. And the LDH inhibitor component can be any synthesized or natural chemical, which is intended for the purpose of inhibiting LDH to treat any type of cancer. If the LDH inhibitor is a polyphenolic compound, it can further include phenolic acids (benzoic acid or cinnamic acid derivatives), benzofurans, chromones, coumarins, phenylacetic acids, phenols, phenylpropanoids, xanthones, stilbenes, quinones and flavonoids or corresponding derivatives, analogues and pro-drugs (Naczk and Shahidi, Chromatogr A. 2004 Oct. 29; 1054(1-2):95-111). If the LDH inhibitor is a flavonoid, it may be further characterized in that the structure is a aurone, flavone, isoflavone, flavanone, isoflavanone, catechin, flavan, flavanonol, chalcone, anthocyanidin, anthocyanin, proanthocyanidin, flavanol, flavonol, isoflavonol or biflavonoid moiety or corresponding derivatives, analogues and pro-drugs. One skilled in the art of bioflavonoids will recognize that a large number of compounds, both glycosides and aglycones, also fall within the scope of the present invention (Prasain et al., Free Radic Biol Med. 2004 Nov. 1; 37(9):1324-50; Kris-Etherton et al., Am J. Med. 30, 71S-88S. 2002). And while morin was selected based on LDH specificity, other flavonoids such as epigallocatechin gallate and quercetin, as well as thiol oxidizing agents can effectively inhibit LDH, and may be substituted for/or combined with morin. It should be understood that the LDH (−) compound of the present invention can be administered in any pharmaceutically acceptable form including, salts, esters, ethers, derivatives and analogues thereof. The LDH (−) component may also be an extract of/or any form of/or any chemical constituent (s) inherent to any plant species of myrrh, rosemary or black walnut and combinations thereof. It was also noted that extract of sage (Salvia officinalis), clove (Syzygium aromaticum), nutmeg (Myristica fragrans) licorice (Glycyrrhiza uralensis), corriander seed (Coriandrum sativum), eucalyptus leaf (Eucalyptus globules), cinnamon (Cinnamomum cassia), ginger root (Zingiber officinale), and green tea also effectively inhibited LDH enzyme activity. Therefore, either whole extracts or chemical constituents inherent to these herbs can also be incorporated, substituted for/or combined as the LDH (−) component. Whole herbal components can further be prepared by extraction or drying procedures. Any portion of the plant can be used, not limited to the root, seed, nut, stalk, bark, vegetable, fruit, hull, bud, leaf, flower, bulb or entire plant. Pure fresh herbs are typically dried at very low temperature, and macerated into an extract, comprised of one or more of the following: grain alcohol, distilled water, glycerine or vinegar. These also include any liquid, chemical, alcohol, lipophilic oil based solvents or acetone. Depending upon the strength of the herbal extract, dry herb menstrumm ratios can vary (w/v) between 1:5-4:5. Typically herbal extracts are stored in a sterile closed container (glass or suitable), in a warm dry area, away from light for about 0.5-2 weeks with intermittent stirring. The extract is then filtered to remove particulates and stored at a cool temperature in an amber container to prevent exposure to light. While any suitable LDH inhibitor can be used or substituted in the formulation, the following chemicals derived from specific extracts may be further evaluated for LDH inhibition and optionally selected.

Optional active chemical constituents within myrrh may include but are not limited to: cresol, cadinene, campesterol, lindestrene, heerabomyrrhol, commiferin, furanodiene, a-bisabolene, a-commiphoric acid, lindestrene, a-heerabomyrrhol, a-amyrone, germacrene, b-pinene, isofuranogermacrene, cinnamaldehyde, elemol, eugenol, cuminaldehyde, b-bourbonene, b-elemene, curzerenone, furanodienone, γ-bisabolene, heerabolene. gamma-elemene, beta-bourbonene, beta-elemene, isofuranogermacrene, germacrene, furanoeudesma-1,4-diene, furanoeudesma-1,3-diene, 2-methoxy furanodiene, 3-epi-alpha-amyrin, 4-o-methyl-glucuronic-acid, cumic-alcohol, heeraboresene, n-nonacosane or whole extracts of myrrh as processed under any procedure.

Optional active chemical constituents within rosemary may include but are not limited to: cineole, a-humulene, a-pinene, a-terpinol, 5-hydroxy-4′,7-dimethoxyflavone, apigenin, bomeol, caffeic acid, calacorene, carvone, camosol, caproic acid, camphor, camphene, calamenene, eugenol, myrcene, chlorogenic acid, nopol, nepetrin, picrosalvin, piperitenone, b-elemene, b-fenchene, diosmin, cadalene, bomylene, cineole, cirsilion, cadinene, diosmetin, a-bisabolol, eriodictiol, eudesmol, γ-muurolol, genkwanin, methoxy-rosmanol, a-amorphene, a-amyrin, a-fenchene, a-selinene, apigenin-7-glucoside, hesperidin, limonene, luteolin, rosmadial, rosmanol, rosmaric acid, rosemaricine, rosmarinic acid, rosmarinol, rosmariquinone, safrole, salvigenin, thymol, anethole, carveol, myrtenol, pinocarveol, ursolic acid, verbenol, verbenone, zingiberene, b-carotene, geraniol, 7-b-amyrenone, 7-ethoxy-rosmanol, hispidulin, isoborneol, isopinocarveol, isorosmanol, isorosmaricine, labiatic acid, ledene, linalol, luteolin-7-glucoside or whole extracts of rosemary as processed under any procedure.

Optional active chemical constituents within black walnut may include but are not limited to: 2-methyl, 1,4-napthoquinone, 2,3-dihydro-5-hydroxy-2-methyl-1,4 napthalenedione, 5-hydroxy-2-methyl-1,4-napthoquinone, 5-hydroxy-3-methyl-1,4-napthoquinone, 2,3-dimethyl-5-hydroxy-1,4-napthoquinone, and 2,3-dihydro-5-hydroxy-1,4-napthalenedione, 1,4-napthoquinone or whole extracts of black walnut as processed under any procedure.

The formulation may comprise one or more of a combination of the aforementioned to optimize efficacy, however the following are some examples as would apply to humans. The term “OXPHOS (+)” represents mitochondrial augmenting component, “LDH (−)” represents the LDH inhibitor component and “AIC (−)” represents a compound other than DMBQ capable of inhibiting the metabolic enzymes or pathways as previously defined. DMBQ or the AIC (−) can be present at between 0-100%, the broad range is not necessarily limited by the upper limit and the “*” represents components of the formulation that were used in animals or humans.

### EXAMPLE 1

| | Broad | | Narrow | |
|---|---|---|---|---|
| Constituents | Range | Units | Range | Units |
| OXPHOS (+) | | | | |
| Ubiquinone (50) | 0-1000+ | Mgs/day/ | 200-400 | Mgs/day/ |
| | | human | | human |
| Riboflavin * | 0-1000+ | Mgs/day/ | 100-400 | Mgs/day/ |
| | | human | | human |
| LDH (−) | | | | |
| Rosemary Extract | 0-25+ | Mls/day/ | 10-20 | Mls/day/ |
| | | human | | human |
| Morin * | 0-1000+ | Mgs/day/ | 100-400 | Mgs/day/ |
| | | human | | human |
| Myrrh Extract | 0-25+ | Mls/day/ | 10-20 | Mls/day/ |
| | | human | | human |
| DMBQ * ± Q(1-3) ± | 0-1000+ | Mgs/day/ | | |
| AIC (−) | | human | | |
| ±FDA approved | | | | |
| chemotherapy drug | | | | |

* Pilot tested against mammary carcinoma in Nude Mice - comparable to taxol - no observable side effects

### EXAMPLE 2

| | Broad | | Narrow | |
|---|---|---|---|---|
| Constituents | Range | Units | Range | Units |
| OXPHOS (+) | | | | |
| Riboflavin * | 0-1000+ | Mgs/day/ | 100-400 | Mgs/day/ |
| | | human | | human |
| LDH (−) | | | | |
| Rosemary Extract * | 0-25+ | Mls/day/ | 10-20 | Mls/day/ |
| | | human | | human |
| Myrrh Extract * | 0-25+ | Mls/day/ | 10-20 | Mls/day/ |
| | | human | | human |
| ±DMBQ ± (Q1-3) ± | 0-1000+ | Mgs/day/ | | |
| AIC(−) | | human | | |
| ±FDA approved | | | | |
| chemotherapy drug | | | | |

* Preliminary findings in humans ± chemotherapy indicated the combination to exhibit anti-cancer effects and blocked the side effects of standard chemotherapy. Future research will be required to substantiate these findings.

### EXAMPLE 3

| | Broad | | Narrow | |
|---|---|---|---|---|
| Constituents | Range | Units | Range | Units |
| OXPHOS (+) | | | | |
| Ubiquinone (50) | 0-1000+ | Mgs/day/ | 200-400 | Mgs/day/ |
| | | human | | human |
| Riboflavin | 0-1000+ | Mgs/day/ | 100-400 | Mgs/day/ |
| | | human | | human |
| LDH (−) | | | | |
| Rosemary Extract | 0-25+ | Mls/day/ | 10-20 | Mls/day/ |
| | | human | | human |
| Morin | 0-1000+ | Mgs/day/ | 100-400 | Mgs/day/ |
| | | human | | human |
| Myrrh Extract | 0-25+ | Mls/day/ | 10-20 | Mls/day/ |
| | | human | | human |
| ± Extracts of one | 0-25+ | Mls/day/ | 10-20 | Mls/day/ |
| or more of | | human | | human |
| Nutmeg, Clove, | | | | |
| Cinnamon, Ginger, | | | | |
| Corriander | | | | |
| ±DMBQ ± Q(1-3) ± | 0-1000+ | Mgs/day/ | | |
| AIC (−) | | human | | |
| ±FDA approved | | | | |
| chemotherapy drug | | | | |

### EXAMPLE 4

| | Broad | | Narrow | |
|---|---|---|---|---|
| Constituents | Range | Units | Range | Units |
| OXPHOS (+) | | | | |
| Ubiquinone (50) | 0-1000+ | Mgs/day/ | 200-400 | Mgs/day/ |
| | | human | | human |
| Folic Acid (Folate) | 0-1000+ | Mgs/day/ | 0.2-1 | Mgs/day/ |
| | | human | | human |
| B-3 (Niacin) | 0-500+ | Mgs/day/ | 100-400 | Mgs/day/ |
| | | human | | human |
| B-6 (Pyridoxine) | 0-1000+ | Mgs/day/ | 100-400 | Mgs/day/ |
| | | human | | human |
| B-12 (Cobalamin) | 0-10+ | μgs/day/ | 50-400 | μg/day/ |
| | | human | | human |
| C (Ascorbate) | 0-1000+ | Mgs/day/ | 100-400 | Mgs/day/ |
| | | human | | human |
| Magnesium | 0-400+ | Mgs/day/ | 100-400 | Mgs/day/ |
| | | human | | human |
| B-5 (Pantothenate) | 0-1000+ | Mgs/day/ | 100-400 | Mgs/day/ |
| | | human | | human |
| B-2 (Riboflavin) | 0-1000+ | Mgs/day/ | 100-400 | Mgs/day/ |
| | | human | | human |
| B-1 (Thiamin) | 0-100+ | Mgs/day/ | 10-100 | Mgs/day/ |
| | | human | | human |
| Biotin | 0-400+ | μgs/day/ | 100-400 | μgs/day/ |
| | | human | | human |
| Lipoic Acid | 0-1000+ | Mgs/day/ | 100-400 | Mgs/day/ |
| | | human | | human |
| LDH (−) | | | | |
| Rosemary Extract | 0-25+ | Mls/day/ | 10-20 | Mls/day/ |
| | | human | | human |
| ±Extracts of one or | 0-25+ | Mls/day/ | 10-20 | Mls/day/ |
| more of Nutmeg, | | human | | human |
| Clove, Cinnamon, | | | | |
| Ginger, Corriander | | | | |
| Morin | 0-1000+ | Mgs/day/ | 100-400 | Mgs/day/ |
| | | human | | human |
| Myrrh Extract | 0-25+ | Mls/day/ | 10-20 | Mls/day/ |
| | | human | | human |
| ±DMBQ ± Q(1-3) ± | 0-1000+ | Mgs/day/ | | |
| AIC(−) | | human | | |
| ±FDA approved | | | | |
| chemotherapy drug | | | | |

The types of tumor treated by the formulation can be that of any organ, tissue or cell, including benign and malignant, and in humans or any species of animal. More specifically, the formulation may potentially be used to treat or prevent many types of cancers including but not limited to: cancer of the skin, breast, colon, kidney, bone, blood, lymph, stomach, gastrointestinal, ovary, prostate, liver, lung, head and neck, gallbladder, adrenal, brain, central nervous system, bronchial, eye, hypothalamus, parathyroid, thyroid, pancreas, pituitary, nose, sinus, mouth, endometrium, bladder, cervical, bile duct and specific types such as acute lymphoblastic leukemia, acute myeloid leukemia, AIDS related cancers, Burkitt's lymphoma, astrocytomas/gliomas and Hodgkin's lymphoma.

The term “pharmaceutically acceptable carrier” is defined as any safe material that acts as a vehicle for delivery including but not limited to: water, saline, starches, sugars, gels, lipids, waxes, paraffin derivatives, glycerols, solvents, oils, proteins, talc, glycols, electrolyte solutions, alcohols, gums, fillers, binders, cellulose, magnesium stearate, emulsifiers, humectants, preservatives, buffers, colorants, emollients, foaming agents, sweeteners, thickeners, surfactants, additives, solvents, lubricants or the like. The pharmaceutically acceptable carrier includes one or more compatible solid or liquid filler diluents or encapsulating substances that are suitable for administration to humans or animals.

The form of a pharmaceutically acceptable carrier used to deliver the treatment to a human or animal is all inclusive not limited to a cream, solid, liquid, powder, paste, gel, tablet, granule, foam, pack, ointment, aerosol, solvent, tablet, diluent, capsule, pill, drink, liposome, syrup, solution, suppository, emulsion, suspension, dispersion, food, bolus, electuary, paste or other bio-delivery system or agent. Formulations of the present invention embodiments include pharmaceutically acceptable carriers and delivery systems adapted for varying route of administration such as topical, enteral and parenteral including but not limited to: oral, rectal, nasal, vaginal, subcutaneous, intramuscular, intravenous, intratumor, intraperitoneal, intramammary, intraosseous infusion, transmucosal, transdermal, epicutaneous, intracutaneous, epidural, intrathecal, inhalation, opthalamic or other suitable route. Formulations for parenteral administration include aqueous and non-aqueous isotonic sterile solutions, which may contain anti-oxidants, oils, glycols, alcohols, buffers, bacteriostats, solutes, suspending agents, biodegradable time-release polymers, surfactants, preservatives and thickening agents. Formulations of the present invention adapted for oral administration may contain a predetermined quantity of the active ingredient and take the form of sprays, liquids, syrups, beverages, capsules, powders, granules, solutions, suspensions, tablets, food, lozenges or any other form in which the active ingredients are taken by mouth and absorbed through the alimentary canal. Enteral formulations may also incorporate the active ingredients with pharmaceutically acceptable carriers such as buffers, gums, surfactants, fillers, preservatives, bulking agents, colorants, diluents, flavoring agents, emulsifiers, sugars, oils, cellulose, gelatin, flour, maltodextrose, time release polymers and the like.

The term “therapeutically effective amount” is defined as an amount of one or more of the active ingredients that comprise this invention, administered to an animal or human at a dose such that efficacy of the treatment can bring about remission, prevention or halting of tumor growth or any other desired clinical result. The formulation may be presented in unit dosage form and may be prepared by any method well known in the art of pharmacy. The active ingredients of the formulation may be presented in liquid or solid, in ampoules or vials (preferably amber) or pill form and can be further incorporated with a pharmaceutically acceptable carrier, appropriate for the method of delivery as deemed appropriate by one skilled in the art.

The formulation can be administered alone or in combination to augment any chemotherapy agent(s) including but not limited to: acetogenins, actinomycin D, adriamycin, aminoglutethimide, asparaginase, bleomycin, bullatacin, busulfan, carmustine, carboplatin, chlorambucil, cisplatin, cyclophosphamide, cytarabine, dacarbazine, daunorubicin, doxorubicin, epirubicin, estradiol, etoposide, fludarabine, flutamide, fluorouracil, floxuridine, gemcitabine, glaucarubolone, hexamethylmelamine, hydroxyurea, idarubicin, ifosfamide, interferon, irinotecan, leuprolide, lomustine, mechlorethamine, melphalan, mercaptopurine, methotrexate, mitomycin, mitozantrone, mitotane, oxaliplatin, pentostatin, plicamycin, procarbazine, quassinoids, simalikalactone, steroids, streptozocin, semustine, tamoxifen, taxol, taxotere, teniposide, thioguanine, thiotepa, tomudex, topotecan, treosulfan, vinblastine, vincristine, vindesine and vinorelbine or mixtures thereof.

The formulation of substances that comprises this invention are not necessarily limited to definition by mechanism, since these agents may also meditate tumoricidal effects through other various means. On the other hand, the invention discloses a means through a mechanism to treat or prevent cancer, by specifically and intentionally creating a formulation that combines one or more compounds classified under OXPHOS (+), AIC (−) and/or DMBQ and LDH (−). The mechanism of manipulating glucose metabolism in cancer cells through the described approach comprises this invention, and also includes any or all type of modifications or methods to the development of a formula to achieve these means, that are obvious to one skilled in the art, but not described in the aforementioned and adhere to the scope of the invention.

What is claimed is:

1. A composition comprising:
A therapeutically effective amount of one or more of the following;
(a) any constituent chemical(s), substance(s), agent(s) or plant extract(s) and mixtures thereof, that are capable of augmenting oxidative phosphorylation or mitochondrial respiration, herein termed “OXPHOS (+)”;
(b) any constituent chemical(s), substance(s), agent(s) or plant extract(s) and mixtures thereof that can inhibit lactic acid dehydrogenase herein termed “LDH (−)”;
(c) any constituent chemical(s), substance(s), agent(s) or plant extract(s) and mixtures thereof, that can inhibit one or more of the following: malate synthase, isocitrate lyase, phosphoenolpyruvate carboxylase/carboxykinase, glycolate oxidase, phosphoglycolate phosphatase, glycolaldehyde dehydrogenase, pyruvate carboxylase, citrate lyase, aconitase, acetate-coA ligase, ferridoxin oxidoreductase, fructose 1,6-bisphosphatase, 2,3-diphosphoglycerate mutase, propionyl CoA carboxylase, malic enzyme, acetyl CoA carboxylase and ribulose-1,5-bisphosphate carboxylase, herein termed anaerobic inhibiting component (AIC (−)), wherein said AIC (−) can further comprise 2-3-dimethoxy-5-methyl-1,4 benzoquinone and/or ubiquinones (5-45), wherein said 2-3-dimethoxy-5-methyl-1,4 benzoquinone and ubiquinones include chemical derivatives, analogs and mixtures thereof, and;
(d) optionally, one or more chemotherapy drug(s) used for the treatment of cancer and/or a pharmaceutically acceptable carrier.

2. A composition of claim 1 wherein said OXPHOS (+) further comprises any constituent (s) that can augment or contribute to the function of NADH:ubiquinone oxidoreductase (complex I), succinate dehydrogenase-CoQ oxoreductase (complex II), ubiquinol:cytochrome c oxidoreductase (complex III), cytochrome c oxidase (complex IV), ATP synthase (complex V) or mitochondrial respiratory function either directly or indirectly such as metabolic precursors or compounds required for the biosynthesis of coenzyme Q10, Krebs cycle or respiratory enzymes or the function thereof; and said LDH (−) can comprise any constituent (s) that are capable of inhibiting any relevant isoform of the LDH enzyme, albeit preferably LDH-V, wherein said LDH can be derived from any relevant source such as plant, bacteria, yeast, mold, fungus, animal or tumor.

3. A composition according to claim 1, in which said OXPHOS (+) is further comprised of one or more selected from the group consisting of riboflavin, flavin mononucleotide, flavin adenine dinucleotide, 5-amino-6-(5′-phosphoribitylamino)uracil, 6,7-Dimethyl-8-(1-D-ribityl)lumazine, ribitol, 5,6-dimethylbenzimidazole, pharmaceutically acceptable salts, precursors and derivatives of the vitamin B2 molecule and ubiquinone (50); and said LDH (−) is 2′,3,4′5,7-pentahydroxyflavone or an analogous alternative.

4. A composition according to claim 3, wherein said analogous alternative further comprises one or more selected from the group consisting of extract solution(s) or solids derived from rosemary, myrrh, blackwalnut, sage, nutmeg, ginger, clove, cinnamon, green tea, corriander, eucalyptus and chemical constituents inherent to the aforementioned, a polyphenolic compound, citric acid, epigallocatechin gallate and quercetin.

5. A composition according to claim 1, wherein said chemical derivatives further comprise synthetic or natural derivatives of 2,3-dimethoxy-5-methyl-1,4-benzoquinone or ubiquinones (5-45); and said analogs further comprise hydroquinones, ubichromenols (0-45), ubichromanols (0-45) and ubiquinols (0-45).

6. A composition according to claim 2, wherein said precursors further comprise para-hydroxybenzoate, para-hydroxycinnamate or para-hydroxyphenylpyruvate, para-hydroxyphenyllactate, polyprenyl-para-hydroxybenzoate, tyrosine, phenylalanine and isopentyl-diphosphate or mixtures thereof; said compounds further comprise tetrahydrobiopterin, vitamins B2, B6, B12, folate, niacin, vitamin C and pantothenic acid and mixtures thereof and said OXPHOS (+) further comprises vitamin B1, lipoic acid and biotin.

7. A composition according to claim 1, wherein said pharmaceutically acceptable carrier is further comprised of water, saline, starches, sugars, gels, lipids, waxes, glycerol, solvents, oils, liquids, proteins, glycols, electrolyte solutions, alcohols, fillers, binders, emulsifiers, humectants, preservatives, buffers, colorants, emollients, foaming agents, sweeteners, thickeners, surfactants, additives and solvents and mixtures thereof.

8. A composition according to claim 7, wherein said pharmaceutically acceptable carrier is made suitable for oral, injectable or external administration and further comprises the form of a solid, liquid, powder, paste, gel, tablet, granule, foam, pack, aerosol, solvent, diluent, capsule, pill, drink, liposome, syrup, solution, suppository, emulsion, enema, suspension, dispersion, food, bio-delivery agents and mixtures thereof.

9. A composition according to claim 1 further comprising one or more selected from the group consisting of 2-3-dimethoxy-5-methyl-1,4 benzoquinone, ubiquinone (s) (5-45), corresponding derivatives and analogues present at about 0-100% wt of total composition, wherein said OXPHOS (+) comprises one or more selected from the group consisting of riboflavin, flavin mononucleotide, flavin adenine dinucleotide and derivatives of the vitamin B2 molecule and ubiquinone (50) and is present at about 0-100% wt of total composition and said LDH (−) further comprises one or more selected from the group consisting of 2′,3,4′5,7-pentahydroxyflavone, citric acid, a polyphenolic compound, epigallocatechin gallate, quercetin, extract solution(s) or solids derived from rosemary, myrrh, blackwalnut, green tea, sage, nutmeg, clove, cinnamon, ginger, corriander, eucalyptus and chemical constituents inherent to the aforementioned and is present at about 0-100% wt of total composition.

10. A composition according to claim 9 further comprising 2-3-dimethoxy-5-methyl-1,4 benzoquinone, ubiquinone (s) (5-45), corresponding derivatives and analogues present at about 30-80% wt of total composition, wherein said OXPHOS (+) is present at about 15-30% wt of total composition and said LDH (−) is present at about 10-15% wt of total composition.

11. A composition according to claim 9, wherein said OXPHOS (+) is present at a concentration between 10 to 40% wt of total composition and said LDH (−) is present at a concentration between about 60 to 90% wt of total composition.

12. A method of preventing or treating cancer comprising administering to a patient in need, a therapeutically effective amount of one or more of the following;
(a) any constituent chemical(s), substance(s), agent(s) or plant extract(s) and mixtures thereof, that are capable of augmenting oxidative phosphorylation or mitochondrial respiration, herein termed “OXPHOS (+)”;
(b) any constituent chemical(s), substance(s), agent(s) or plant extract(s) and mixtures thereof that can inhibit lactic acid dehydrogenase herein termed “LDH (−)”;
(c) any constituent chemical(s), substance(s), agent(s) or plant extract(s) and mixtures thereof, that can inhibit one or more of the following: malate synthase, isocitrate lyase, phosphoenolpyruvate carboxylase/carboxykinase, glycolate oxidase, phosphoglycolate phosphatase, glycolaldehyde dehydrogenase, pyruvate carboxylase, citrate lyase, aconitase, acetate-coA ligase, ferridoxin oxidoreductase, fructose 1,6-bisphosphatase, 2,3-diphosphoglycerate mutase, propionyl CoA carboxylase, malic enzyme, acetyl CoA carboxylase and ribulose-1,5-bisphosphate carboxylase, herein termed anaerobic inhibiting component (AIC (−)), wherein said AIC (−) can further comprise 2-3-dimethoxy-5-methyl-1,4 benzoquinone and/or ubiquinones (5-45), wherein said 2-3-dimethoxy-5-methyl-1,4 benzoquinone and ubiquinones include chemical derivatives, analogs and mixtures thereof, and;
(d) optionally, one or more chemotherapy drug(s) used for the treatment of cancer and/or a pharmaceutically acceptable carrier.

13. The method of claim 12 wherein said OXPHOS (+) further comprises any constituent (s) that can augment or contribute to the function of NADH:ubiquinone oxidoreductase (complex I), succinate dehydrogenase-CoQ oxoreductase (complex II), ubiquinol:cytochrome c oxidoreductase (complex III), cytochrome c oxidase (complex IV), ATP synthase (complex V) or mitochondrial respiratory function either directly or indirectly such as metabolic precursors or compounds required for the biosynthesis of coenzyme Q10, Krebs cycle or respiratory enzymes or the function thereof; and said LDH (−) can comprise any constituent (s) that are capable of inhibiting any isoform of the LDH enzyme, albeit preferably LDH-V, wherein said LDH can be derived from any relevant source such as plant, bacteria, yeast, mold, fungus, animal or tumor.

14. The method of claim 12, in which said OXPHOS (+) is further comprised of one or more selected from the group consisting of riboflavin, flavin mononucleotide, flavin adenine dinucleotide, 5-amino-6-(5′-phosphoribitylamino)uracil, 6,7-Dimethyl-8-(1-D-ribityl)lumazine, ribitol, 5,6-dimethylbenzimidazole, pharmaceutically acceptable salts, precursors and derivatives of the vitamin B2 molecule and ubiquinone (50); and said LDH (−) is 2′,3,4′5,7-pentahydroxyflavone or an analogous alternative.

15. The method of claim 14, wherein said analogous alternative further comprises one or more selected from the group consisting of extract solution(s) or solids derived from rosemary, myrrh, blackwalnut, sage, nutmeg, clove, cinnamon, ginger, green tea, corriander, eucalyptus and chemical constituents inherent to the aforementioned, a polyphenolic compound, epigallocatechin gallate, citrate and quercetin.

16. The method of claim 12, wherein said chemical derivatives further comprise synthetic or natural derivatives of 2,3-dimethoxy-5-methyl-1,4-benzoquinone or ubiquinones (5-45); and said analogs further comprise hydroquinones, ubichromenols (0-45), ubichromanols (0-45) and ubiquinols (0-45).

17. The method of claim 13, wherein said precursors further comprise para-hydroxybenzoate, para-hydroxycinnamate or para-hydroxyphenylpyruvate, para-hydroxyphenyllactate, polyprenyl-para-hydroxybenzoate, tyrosine, phenylalanine and isopentyl-diphosphate or mixtures thereof; said compounds further comprise tetrahydrobiopterin, vitamins B2, B6, B12, folate, niacin, vitamin C and pantothenic acid and mixtures thereof and said OXPHOS (+) further comprises vitamin B 1, lipoic acid and biotin.

18. The method of claim 12, wherein said pharmaceutically acceptable carrier is further comprised of water, saline, starches, sugars, gels, lipids, waxes, glycerol, solvents, oils, liquids, proteins, glycols, electrolyte solutions, alcohols, fillers, binders, emulsifiers, humectants, preservatives, buffers, colorants, emollients, foaming agents, sweeteners, thickeners, surfactants, additives and solvents and mixtures thereof and said administration further comprises one or more of the following routes: parental, oral, topical, intra-venous, intra-arterial, intra-tumor, intra-muscular, intra-peritoneal and subcutaneous.

19. The method of claim 18, wherein said pharmaceutically acceptable carrier is made suitable for oral, injectable or external administration and further comprises the form of a solid, liquid, powder, paste, gel, tablet, granule, foam, pack, aerosol, solvent, diluent, capsule, pill, drink, liposome, syrup, solution, suppository, emulsion, enema, suspension, dispersion, food, bio-delivery agents and mixtures thereof.

20. The method of claim 12 further comprising one or more selected from the group consisting of 2-3-dimethoxy-5-methyl-1,4 benzoquinone, ubiquinone (s) (5-45), corresponding derivatives and analogues present at about 0-100% wt of total composition, wherein said OXPHOS (+) comprises one or more selected from the group consisting of riboflavin, flavin mononucleotide, flavin adenine dinucleotide and derivatives of the vitamin B2 molecule and ubiquinone (50) and is present at about 0-75% wt of total composition and said LDH (−) further comprises one or more selected from the group consisting of 2′,3,4′5,7-pentahydroxyflavone, citric acid, a polyphenolic compound, epigallocatechin gallate, quercetin, extract solution(s) or solids derived from rosemary, myrrh, blackwalnut, green tea, sage, nutmeg, clove, cinnamon, corriander, eucalyptus and chemical constituents inherent to the aforementioned and is present at about 0-100% wt of total composition.

21. The method of claim 20 further comprising 2-3-dimethoxy-5-methyl-1,4 benzoquinone, ubiquinone (s) (5-45), corresponding derivatives and analogues present at about 30-80% wt of total composition, where said OXPHOS (+) is present at about 15-30% wt of total composition and said LDH (−) is present at about 10-15% wt of total composition.

22. The method of claim 20, wherein said OXPHOS (+) is present at a concentration between 10 to 40% wt of total composition and LDH (−) is present at a concentration between about 60 to 90% wt of total composition.

23. The method of claim 12, wherein said cancer further comprises one or more selected from the group consisting of benign and malignant tumors of the skin, breast, colon, kidney, bone, blood, lymph, stomach, gastrointestinal, ovary, prostate, liver, lung, head and neck, gallbladder, adrenal, brain, central nervous system, bronchial, eye, hypothalamus, parathyroid, connective tissue, thyroid, pancreas, pituitary, nose, sinus, mouth, endometrium, bladder, cervical, bile duct, epithelial and specific types such as acute lymphoblastic leukemia, acute myeloid leukemia, AIDS related cancers, Burkitt's lymphoma, astrocytomas/gliomas and Hodgkin's lymphoma.

24. The method of claim 12, wherein said chemotherapy drug(s) further comprise one or more selected from the group consisting of acetogenins, actinomycin D, adriamycin, aminoglutethimide, asparaginase, bleomycin, bullatacin, busulfan, carmustine, carboplatin, chlorambucil, cisplatin, cyclophosphamide, cytarabine, dacarbazine, daunorubicin, doxorubicin, epirubicin, estradiol, etoposide, fludarabine, flutamide, fluorouracil, floxuridine, gemcitabine, glaucarubolone, hexamethylmelamine, hydroxyurea, idarubicin, ifosfamide, interferon, irinotecan, leuprolide, lomustine, mechlorethamine, melphalan, mercaptopurine, methotrexate, mitomycin, mitozantrone, mitotane, oxaliplatin, pentostatin, plicamycin, procarbazine, quassinoids, simalikalactone, steroids, streptozocin, semustine, tamoxifen, taxol, taxotere, teniposide, thioguanine, thiotepa, tomudex, topotecan, treosulfan, vinblastine, vincristine, vindesine and vinorelbine.
